(19) United States
(12) Patent Application Publication
(10) Pub. No.: US 2012/0230918 A1
(43) Pub. Date: Sep. 13, 2012

(54) NON-INVASIVE IN VIVO OPTICAL IMAGING METHOD

(75) Inventors: Michael Dobosz, Penzberg (DE); Werner Scheuer, Penzberg (DE); Steffen Strobel, Penzberg (DE)

(73) Assignee: Hoffmann-La-Roche Inc., Nutley, NJ (US)

(21) Appl. No.: 13/387,515

(22) PCT Filed: Jul. 28, 2010
(86) PCT No.: PCT/EP2010/060957

§ 371 Date: May 22, 2012

(30) Foreign Application Priority Data
Jul. 28, 2009 (EP) 09166531.5

(51) Int. Cl. A61B 5/06

(52) U.S. Cl. 424/9.6

(57) ABSTRACT

The present invention relates to a non-invasive method of determining the presence, quantifying the blood level, and/or monitoring or determining the blood clearance of a fluorescent analyte which comprises a fluorescent entity and a second entity, in the blood of a subject, comprising or consisting of the steps: (a) directing excitation light of at least one predetermined wavelength onto a delineated region comprising at least a portion of the pupil of said subject, to excite the fluorescent entity, (b) receiving light emitted from said fluorescent analyte with a wavelength distinguishable from the predetermined wavelength of (a), through the eye of said subject, thereby determining the presence, quantifying the blood level, and/or monitoring or determining the blood clearance of said fluorescent analyte in the blood of said subject. The present invention further relates to a fluorescent analyte or fluorescent label as defined in any one of the preceding claims for use in any one of the preceding methods. The present invention furthermore relates to the use of a fluorescent analyte or fluorescent label as defined in any one of the preceding claims for the preparation of a diagnostic composition which is to be employed in any one of the preceding methods. The present invention furthermore relates to a device for use in any of the methods defined herein.

The present invention relates to a non-invasive method of determining the presence, quantifying the blood level, and/or monitoring or determining the blood clearance of a fluorescent analyte which comprises a fluorescent entity and a second entity, in the blood of a subject, comprising or consisting of the steps: (a) directing excitation light of at least one predetermined wavelength onto a delineated region comprising at least a portion of the pupil of said subject, to excite the fluorescent entity, (b) receiving light emitted from said fluorescent analyte with a wavelength distinguishable from the predetermined wavelength of (a), through the eye of said subject, thereby determining the presence, quantifying the blood level, and/or monitoring or determining the blood clearance of said fluorescent analyte in the blood of said subject. The present invention further relates to a fluorescent analyte or fluorescent label as defined in any one of the preceding claims for use in any one of the preceding methods. The present invention furthermore relates to the use of a fluorescent analyte or fluorescent label as defined in any one of the preceding claims for the preparation of a diagnostic composition which is to be employed in any one of the preceding methods. The present invention furthermore relates to a device for use in any of the methods defined herein.

Non-invasive imaging can be traced back to the discovery of X-rays by Wilhelm Roentgen in 1895. Modern-day medicine reveals a huge increase in the number of imaging technologies and their applications. Computer tomography (CT), positron emission tomography (PET), single-photon-emission computerized tomography (SPECT) and magnetic resonance imaging (MRI) are some of the classical noninvasive imaging techniques. These technologies allow the diagnosis of diseases, such as cancer, on the basis of anatomical, morphological and physiological changes.

However, most of these established techniques lack sensitivity, reduced specific targeting and are incapable exhibiting functional changes on molecular basis and thus are not appropriate tools in basic-research, preclinical and translational applications. Therefore, diseases can be just diagnosed at late, morphologic visible points in time. For this reason, researcher have focused on molecular-functional imaging technologies for imaging gene expression, receptor activation, signaling pathways, apoptosis and multidrug resistance with high sensitivity and high contrast (Weissleder and Mahmood, Radiology 2001; Vol. 219: 316-333).

In contradistinction to “classical” diagnostic imaging, for example, magnetic resonance (MR), computed tomography (CT), and ultrasound (US) imaging, molecular imaging such as optical molecular imaging analyses molecular abnormalities that are the basis of disease, rather than imaging the end-effects of these molecular alterations.

Optical molecular imaging, such as fluorescence and bioluminescence imaging, is one of the youngest cutting-edge technologies in medical diagnostics and became a powerful tool for imaging changes at the molecular level. The aim of this technology is, to visualize and quantify molecular changes during the development of diseases.

Of particular interest are fluorochromes that emit in the near infrared (NIR), a spectral window, whereas hemoglobin and water absorb minimally so as to allow photons to penetrate for several centimeters in tissue.

The fluorochromes used as labels should absorb and emit light in the near-infrared range. They should reveal a high fluorescence quantum yield, good water solubility and photosensitivity (Heiduschka et al., Investigative Ophthalmology & Visual Science 2007; Vol. 48: 2814-2823). In the past years, cyanine-dyes (Cy-dyes) proved to be effective and reliable fluorescence dyes in biomedical research. Because of their fluorescence in the near-infrared region, photons are allowed to travel deep through the tissue and they are characterized of a low tissue autofluorescence. They are very photostabile and insensitive against pH variations. Besides cyanine-dyes, alexa-dyes are common fluorochromes used in optical imaging techniques.

Diagnosing diseases at early states would be a great advantage for the prognosis of patients and appropriate treatments could be started in time. Functional imaging techniques capabilities include the ability for studying functional pathways, assess angiogenesis and hypoxia at cellular and molecular levels. To visualize biological processes non-invasive and to be able to do quantifications, an injectable imaging agent is required. This probe comprises a label, which can be detected highly sensitive and a ligand exhibiting high affinity towards the desired target. A strategy to reinforce the label specific signal is needed to increase sensitivity and last but not least, a high resolution imaging modality to detect the label specific signal is required (Hengerer and Mertelmeier, Electromedica 2001; Vol. 1: 44-49). Alternative labelling techniques, such as genetic reporters and exogenous cell trackers are based on different labeling strategies, but they have largely been limited to mouse models and basic biological sciences.

Optical molecular technologies are increasingly being used to understand the complexity, diversity and in vivo behaviour of cancer. Tumors can be detected using labelled antibodies specific to extracellular receptors. Gene therapy can be monitored using molecular marker genes. This technology enables an in vitro and/or in vivo evaluation of appropriate target structures and the efficiency of therapeutics against these structures can be determined, allowing an accelerated drug development (Hengerer and Mertelmeier, Electromedica 2001; Vol. 1: 44-49; Högemann and Weissleder, Radiologie 2001; Vol. 41: 116-120; Reiser et al., Lehrbuch der Radiologie, Thieme Verlag, 2004; Cutler, Surg Gunecol Obstet 1929: 721-728). At present, mainly CT, MRT, PET and SPECT are the common imaging technologies for testing the therapeutic efficiency in clinical trials.

Photons travelling through tissue and interacting with tissue components form the basis of optical imaging techniques. Optical imaging, the diagnostic with light, was first reported by Cutler in 1929, but just with the development of high sensitive CCD detection systems and the opportunity of coupling fluorescent dyes with biochemical markers, pushed optical imaging into the focus of researchers (Ntziachristos and Bremer, Radiology 2003; Vol. 1: 195-208). Fluorescent illumination and observation has been one of the most rapidly adapted imaging technologies, in both medicine and biological sciences. Many naturally occurring materials emit light of a particular wavelength when exposed to light with another wavelength. This appearance is called luminescence. If the emitted light occurs rapidly (around one-million of a second), it is defined as fluorescence and if the emission takes longer than one-million of a second, the luminescence is called phosphorescence. This occurrence has been known since a long time and those fluorescence molecules have proven extremely useful as labels in many biological systems. Materials that fluoresce almost always emit light (λemit) at a longer wavelength than the wavelength of the exciting light (λabsorb). The difference between those wavelengths is called the Stoke's Shift (λstokes) and it makes a statement about the energy level (ΔE) between excited and emitted wavelength. A range of excitation wavelength will excite fluorescence. This range is known as absorption spectrum. The emission spectrum also covers a range of wavelength suggesting that one fluorescence material is not restricted to just one excitation wavelength. Many biological materials are naturally fluorescent and in particular, many vitamins, some hormones, and a variety of enzymes and structural proteins. Those molecules often emit fluorescent light strong enough to interfere with specific fluorescence labelling studies in vivo. The so called auto-fluorescence gives an unwanted background and therefore, both the excitation light and emitted light are needed to be highly filtered. Restricting the excitation light wavelengths may reduce the amount of auto-fluorescence. Restricting the wavelength range of the emitted light minimizes the amount of auto-fluorescence that interferes with observing and measuring the desired specific fluorescence. With the development of fluorescent dyes, emitting light in the near-infrared range of the electromagnetic spectrum, the in vivo diagnostic in deeper tissue levels is possible. The sensitivity depends on the amount and localization of the examined marker (Bremer and Ntziachristos, American Journal of Surgery 2001; Vol. 189: 389-392). Whereas visible light is able to penetrate tissue not deeper than some millimeters, near-infrared photons (650-900 nm) travel through tissue much more efficiently in the range of some centimeters. The reason for this phenomenon is the low absorption coefficient from water, melanin and hemoglobin in that wavelength range. Because of this positive tissue properties the near-infrared wavelength range in also called “diagnostic window” (Mahmood and Weissleder, Molecular cancer therapeutics 2003; Vol. 5: 489-496). In the near-infrared spectrum, photons are absorbed at a minimum level and the tissue autofluorescence is reduced, resulting in an optimal target-background ratio (Licha and Riefke, Photochemistry and Photobiology 2000; Vol. 3: 392-398).

Monitoring the functions of a human or animal body is necessary in many situations. Traditionally, blood samples are taken from a subject and constituents have been measured by spectrophotometry, i.e. an optical imaging technique. A spectrophotometer can also be directly applied to measure the constituents in the blood of a subject by bringing it into contact with the subject, for example by using a device such as a modified contact lens systems, i.e. an in vivo optical imaging system.

WO 90/12534, WO 02/071932 and WO 2006/079824 describe a device, in particular, a modified contact lens system for use in real-time monitoring human or bodily functions such as oxygen levels in the blood via the eye. The invention described in these documents focuses on measuring in particular oxygen concentrations via the eye during anaesthesia since the eye provides a more direct method of assessing the conditions in the brain. This is so because the major blood supply to the eye via the ophthalmic artery is a branch of the internal carotid artery. Accordingly, the eye can, so to say, aptly be referred to as the “window” of the brain.

The device described therein is a non-invasive spectrophotometric system which—in contrast to the prior art (U.S. Pat. No. 5,553,617 and U.S. Pat. No. 5,919,132)—is said to direct light along the axis of the eye by being focused in the centre of the plane of the iris, thereby overcoming the shortcomings of the prior art.

The principle of these spectrophotometric techniques is that light is introduced into the eye. This light passes through the eye and is reflected by the retina. The reflected light (from the retina) and the intensity of the reflected light is measured with a photodiode or other light sensor, and the transmittance for this wavelength is then calculated. Since the eye is the only part of the body that is designed to transmit light, thus acts, so to say, as the cuvette for the spectrophotometer.

In vivo molecular imaging is a rapidly advancing field impacting on, for example, clinical diagnostic imaging. Optical molecular imaging is a method in which an optical contrast substance is introduced to or activated within a subject, and the resultant signal due to the optical contrast substance is measurable using an optical detector such as a camera to provide one or more images.

Optical molecular probes are available which can include fluorescent or luminescent dyes, or absorbing substances, and can be used to target and label specific cell types or activate biochemical processes like bioluminescence. Optical molecular imaging, as compared to magnetic resonance imaging (MRI), X-ray or positron-emission imaging (PET), benefits from the fact that such fluorescent, luminescent or absorbing substances can be small, biocompatible molecules.

In vivo optical molecular imaging is typically performed on small animals to study the physiologic, pathologic or pharmacologic effects of various drugs or diseases. Molecular imaging can also be performed on humans, and it is hoped that molecular imaging will eventually provide substantial advances in diagnostic imaging. The benefits of in vivo imaging of small animals are significant because it allows processes and responses to be visualized in real-time in their native environments, and allows longitudinal studies to be performed using the same small animal over time, allowing evaluation of disease progression or response to treatment. Further, in vivo imaging of small animals reduces the number of animals required for a study, and can reduce the variance in studies where disease manifestation varies from animal to animal, such as cancers in situ.

Optical molecular imaging in, for example, small animals harnesses the power of highly specific and biocompatible contrast agents for drug development and disease research. However, the widespread adoption of in vivo optical imaging has been inhibited by its inability to clearly resolve and identify targeted internal organs. Optical tomography and combined X-ray and micro-computed tomography (micro-CT) approaches developed to address this problem are generally expensive, complex or incapable of true anatomical co-registration

Accordingly, Hillman and Moore, Nature Photonics (2007), Vol. 1: 526-530 provided an all-optical anatomical co-registration for molecular imaging of, for example, small animals using dynamic contrast, i.e. a dynamic fluorescence molecular imaging technique. Their technique uses a time series of images acquired after injection of, for example, an inert dye. Differences in the dye's in vivo distribution dynamics allow precise delineation and identification of organs.

Such co-registered anatomical maps permit longitudinal organ identification irrespective of repositioning or weight gain, thereby promising greatly improved accuracy and versatility for studies of orthotopic disease, diagnostics and therapies In sum, highly advance techniques for optical molecular imaging are available which allow the precise delineation and identification of organs. Moreover, many different types of fluorescent including near-infrared fluorescent and luminescent dyes are available which can be applied in optical molecular imaging.

Even more, the eye has already been recognized to be useful as a “window” to at least the brain.

However, in spite of the foregoing efforts and highly advanced techniques and dyes, commercially viable, non-invasive in vivo (real-time) methods and uses for clinical diagnostic applications such as qualitatively or quantitatively determining foreign and natural physiologic substances in the blood of a subject have not yet been developed.

Thus, the technical problem underlying the present invention was to provide means and methods for the non-invasive monitoring and/or quantifying fluorescent analytes in blood in vivo.

The present invention addresses this need and thus provides as a solution to the technical problem embodiments concerning means and methods as well as uses for qualitatively and/or quantitatively determining in vivo and non-invasively fluorescent analytes in the blood of a subject, whereby the fluorescence labeled analyte is determined, quantified or monitored via the eye of the subject by optical molecular imaging.

These embodiments are characterized and described herein and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supercede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Pharmacokinetics describes how the body affects a specific drug after administration. It follows that “Pharmacokinetics” includes the study of the mechanisms of absorption and distribution of an administered drug, the rate at which a drug action begins and the duration of the effect, the chemical changes of the substance in the body (e.g. by enzymes) and the effects and routes of excretion of the metabolites of the drug. Accordingly, pharmacokinetics provides a rational means of approaching the metabolism of a compound in a biological system. For reviews of pharmacokinetic equations and models, see, for example, Poulin and Theil, J Pharm Sci. (2000), Vol. 89: 16-35; Slob et al., Crit. Rev Toxicol. (1997), Vol. 27: 261-272; Haddad et al., Toxicol Lett. (1996), Vol. 85: 113-126; Hoang, Toxicol Lett. (1995), Vol. 79:99-106; Knaak et al., Toxicol Lett. (1995), Vol. 79):87-98; and Ball and Schwartz, Comput Biol Med. (1994), Vol. 24:269-276. In practice, pharmacokinetics is applied mainly to drug substances, though in principle it concerns itself with all manner of compounds ingested or otherwise delivered externally to an organism, such as nutrients, metabolites, hormones, toxins, etc.

Up to the disclosure of the present invention, the “conventional” measurement of pharmacokinetics (PK) was conventionally performed by i.v. or i.p. injection of drugs. At different time points after the application, blood is drawn and drug serum levels are quantified by different analytical methods (e.g. ELISA, SEC, HPLC, liquid chromatography-tandem mass spectrometry using non-labeled or radiolabeled compounds). In order to receive statistically meaningful data, about 3 to 5 mice are typically used for each time point to get serum peak levels (tmax) and the drug serum half-life (t1/2). For one study about 9 to 15 mice (e.g. 7 time points and 3 to 5 mice for each time point with serial blood sampling) are needed. Both tmax and t1/2 are determined by interpolation, which may influence the accuracy of these values (FIG. 1, 2). Mice are sacrificed after termination of the PK study.

In clear contrast thereto, we propose a method and the corresponding devices which allows, non-invasive, continuous real-time monitoring of the level of a fluorescent analyte (e.g. drug levels) in blood, and should the situation arise, in blood and organs simultaneously in one subject (e.g. a mouse) over a desired time period. Quantification of the fluorescent analyte (e.g. the fluorescent labeled drug) by way of taking blood samples is almost not necessary to get the PK data and there is no need to sacrifice the animals. We demonstrated the utility of this approach by evaluating 3 different compounds:

1. Indocyanine green: a fluorescent dye
2. Pamidronate: a fluorescence labeled bisphosphonate
3. Monoclonal antibody against receptor tyrosine kinase labeled with Cy5

We first evaluated the technical feasibility of this new approach by using indocyanine green (ICG) a fluorescent dye. When injected i.v. into mice, ICG is cleared from the circulation in approximately 2 to 4 min (1, 2) and accumulates in the liver (3). Female BALB/c nude mice received inhalation anaesthesia, were placed in the imaging chamber (FIG. 3) and injected i.v. with a dose of 20 μg/200 μl. The fluorescence signal intensity measurements in the eye was started 10 sec before i.v. injection of ICG and images were recorded every second with an acquisition time of 500 ms over a period of 8 minutes. ICG was excited with light at a wavelength range from 671 to 705 nm and the emission was detected at 820 nm. The highest value of the fluorescence signal intensity in the eye region was normalized to 100 and data depicted in FIG. 5, 6 demonstrate that tmax was reached at 2 min and the half life of the fluorescence intensity was at 6.6 min. In a second experiment, a BALB/c nude mouse with a s.c. growing tumor (Calu3) was injected with ICG i.v. and signal intensity in eye, liver, kidney, brain and the tumor region was monitored. In this experiment tmax was 1.2 min. The signal intensity declined thereafter (t1/2=5.4 min) and accumulation in the liver was observed reaching a plateau at 3.8 min (FIG. 7). These results are in accordance with published data. After successful completion of the feasibility study shown in Example 1, we evaluated a fluorescence labeled bisphosphonate (Pamidronate). Bisphosphonates (e.g. Pamidronate; MW 279) are clinically useful for the treatment of bone disorders. Pamidronate (after i.v. injection) has a serum half life in the range of 20 to 30 min and the bone (tibia) contained the highest concentration of all the tissues examined. Pongchaidecha M et al. Clearance and tissue uptake following 4-hour and 24-hour infusions of pamidronate in rats. Drug Metab Dispos 1993; 21(1):100-104 Daley-Yates et al. A comparison of the pharmacokinetics of 14C-labelled ADP and 99 mTc-labelled ADP in the mouse. Calcif Tissue Int 1988; 43:125-127. We used a fluorescence labeled Pamidronate to calculate tmax and t1/2 plasma levels by measuring the fluorescence intensity in the eye of mice and whole body imaging to monitor the described kinetics. After i.v. injection, Pamidronate has a serum half life in the range of 20 to 30 min and the bone (tibia) contained the highest concentration of all the tissues examined (4, 5). OsteoSense (2 nMol in 200 μl PBS) was injected i.v. and fluorescence signal intensity was recorded every five seconds (acquisition time: 3000 ms; excitation wavelength: 615 to 665 nm; emission wavelength: 780 nm). Serum t1/2 was 34 min (FIG. 7, 8) and accumulation in spine and hind leg is clearly demonstrated at 4.4 hrs and 48 hrs thereafter (FIG. 9). Both observations correlate with published data. Finally, t1/2 of a non-labeled and Cy5 labeled monoclonal antibody targeting receptor tyrosine kinase was compared. Conventional measurements revealed a t1/2 of 7.7 hrs at a dosage of 5 mg/k i.v. (FIG. 10). Using optical imaging t1/2 was 3.05 hrs at a dosage of 2.5 mg/kg i.v. (FIG. 11). These results demonstrate that tmax and t1/2 can be easily performed by simply measuring the fluorescence signal intensities in the eye of anesthesized animals. In contrast to the conventional technique this new approach improves the performance of PK studies since quantification of the drug and data interpolation is not necessary. Furthermore, the number of mice is significantly reduced and mice need not to be sacrificed. Information regarding the accumulation of the drug and t1/2 values from different organs can be obtained time-resolved and on-line. Taking together, this procedure allows multiple measurements in one animal (improving the accuracy of the tmax and t1/2). Compared to conventional methods, work time is significantly reduced, mixing up of blood samples is prevented and the use of non-radioactive materials permits further analysis by routine laboratory methods without the precautions needed with radiochemicals. In addition to tmax and t1/2, organ distribution can be followed up. Such simultaneous measurements facilitate information regarding accumulation in the organ under question compared with t1/2 in serum (e.g. indication of blood brain barrier penetration). Drugs (low molecular weight substances, peptides, proteins, antibodies and siRNA) can be labeled easily with different organic fluorescence dyes. However, before performing such in vivo studies with labeled drugs, functional assays must demonstrate that there is no difference compared to the non-labeled drug. Regarding Hemojuvelin, in vitro studies confirmed that non-labeled and Cy5-labeled Hemojuvelin did not differ in their ability to block BMP-2 induced upregulation of Hepcidin mRNA in HepG2 cells. Also, Biacore data reveal that Cy5-labeled Herceptin has the same binding characteristics compared to non-labeled Herceptin and binds to Her2 expressing tumor cells. The labeled antibody targeting receptor tyrosine kinase still leads to internalization of the receptor. Since animals are not sacrificed, multiple applications of the same and/or another drug (labeled with a fluorochrome with a emission spectra different from the first one) can be applied to get information on drug-drug interactions. Furthermore, new designed drug formulations and optimization of drug dosage after i.v., i.p., oral, inhalation, nasal and dermal applications can be evaluated in normal and in genetically engineered mice (e.g. FcRn knock-outs or hu FcRn transgenics). The extraordinary progress of imaging methods allows the visualization of the performance of drugs and drug delivery systems under in vivo conditions. Detailed and quantitative information about the location and concentration of the drug can be obtained as a function of time, thereby enabling a more profound understanding of biological effects. This information is crucial to the design of optimized drugs.

Therefore, the impact of the new non-invasive imaging methods provided by the present invention is significant. First, the new methods can be used to assess the pharmacokinetics of fluorescent analytes such as fluorescently labeled drugs in real-time and in vivo. This, in turn, is expected to have an impact in drug development, drug testing, and choosing appropriate therapies and therapy changes for a given subject (for example a human patient) and optimization of drug dosage. It is for example conceivable that a patient dependent therapy can be established, i.e. based on the pharmacokinetics of a given set of drugs or drug formulations it is possible to find the best possible medication for each individual patient, i.e. for personalized medication. In fact, the pharmacokinetic profile can be evaluated in each single subject in real time, allowing a fast feedback, in a non-invasive fashion and, therefore, for an optimized medication for each single subject, depending for example on the subject's characteristics such as weight, sex, age, state of health, course of disease etc.

Second, the new molecular imaging/quantitation methods and devices of the invention enable one to study the pharmacokinetic of drugs of any kind in the intact microenvironment of living systems. The new imaging devices, uses and methods will have broad applications in a wide variety of novel biologic, immunologic, and molecular therapies designed to promote the control and eradication of numerous different diseases including cancer, cardiovascular, neurodegenerative, inflammatory, infectious, and other diseases. Furthermore, the described detection systems and methods will have broad applications for seamless disease detection and treatment in combined settings.

Third, the new methods and devices can detect the presence of pathogenic agents, that form the basis of many diseases, not only in vivo but also in real-time.

Fourth, the new methods can be used in pharmacodynamics is sometimes abbreviated as “PD”, and when referred to in conjunction with pharmacokinetics can be referred to as “PKPD”. Pharmacodynamics is the study of the physiological effects of drugs on the body or on microorganisms or parasites within or on the body and the mechanisms of drug action and the relationship between drug concentration and effect.

Optical imaging is a non-invasive and non-ionizing modality that is emerging as a diagnostic tool for different applications. This techniques offer simplistic while highly sensitive modalities for molecular imaging research. Non-invasive visualization of peak drug levels, half life in blood and accumulation to organs can be performed by using fluorescence labelled analytes. Measurements can be performed serially, thus giving the possibility of PK analysis with a temporal resolution in the order of seconds. By multiple measurements (acquisition time is normally below 1 sec) over a time period of several hrs “kinetic movies” can be created allowing to calculate serum peak levels, the half life time in blood, the theoretical concentration immediately after application and the distribution/accumulation into different organs.

Thus, in a first aspect, the present invention provides a non-invasive method of determining the presence of a fluorescent analyte which comprises a fluorescent entity and a second entity, in the blood of a subject, comprising or consisting of the steps:
(a) directing excitation light of at least one predetermined wavelength onto a delineated region comprising at least a portion of the pupil of said subject, to excite the fluorescent entity,
(b) receiving light emitted from said fluorescent analyte with a wavelength distinguishable from the predetermined wavelength of (a), through the eye of said subject, thereby determining the presence of said fluorescent analyte in the blood of said subject.

“Determining the presence” means the qualitative detection of the fluorescent analyte in the blood (or the blood circulation system) of the subject, thereby allowing to determine whether a fluorescent analyte is there (in the blood and/or blood circulation system) or not.

In a further aspect, the present invention provides a non-invasive method of quantifying the blood level of a fluorescent analyte which comprises a fluorescent entity and a second entity, in a subject, comprising the steps of:
(a) directing excitation light of at least one predetermined wavelength onto a delineated region comprising at least a portion of the pupil of said subject, to excite the fluorescent entity,
(b) receiving light emitted from said fluorescent analyte with a wavelength distinguishable from the predetermined wavelength of (a), through the eye of said subject, thereby quantifying the blood level of a fluorescent analyte.

To this end, it is envisaged to administer a defined amount of the analyte in question to the subject, to take blood samples from said subject in order to quantify the amount of said analyte in the blood, and, subsequently, to correlate/compare these data with the fluorescence signal which is obtained by the methods of the present invention (obtained either simultaneously or consecutively). It will be understood that once that correlation took place, it is no longer necessary to take blood samples from the very subject, i.e. once the data are correlated, it is possible to quantify the blood level of a fluorescent analyte by the methods of the present invention.

Alternatively and/or additionally, it is also envisaged to compare the fluorescent signal obtained by the methods of the present invention with serum level data which are already known (e.g. published in the prior art) or which have been evaluated before or afterwards by conventional methods (including blood samples).
Thus, in a preferred embodiment, said light received in step (b) is compared with a reference value, thereby:
   (i) quantifying the blood level of said fluorescent analyte.

The present invention also provides a non-invasive method of monitoring or determining the blood clearance of a fluorescent analyte which comprises a fluorescent entity and a second entity, in a subject, comprising the steps of:
(a) directing excitation light of at least one predetermined wavelength onto a delineated region comprising at least a portion of the pupil of said subject, to excite the fluorescent entity,
(b) receiving light emitted from said fluorescent analyte with a wavelength distinguishable from the predetermined wavelength of (a), through the eye of said subject, thereby monitoring or determining the blood clearance of said fluorescent analyte.

In a preferred embodiment, said light received in step (b) is compared with a reference value, thereby

(ii) determining the blood clearance of said fluorescent analyte.

The term “blood clearance” includes the determination and/or monitoring of the biological half-life, tmax and/or t1/2”.

The term “biological half-life” means the time it takes for an analyte to lose half of its activity, for example, biological, pharmacologic or physiologic activity.

The term “t max” when used herein is the time to reach maximum blood concentration. The maximum blood concentration is the amount of a compound present in the blood of a subject.

“T ½” is the time required for the total amount of an analyte in the body or the concentration of the substance in the blood to decrease by one-half. t ½ can also be used to determine how long it will take to effectively eliminate the analyte from the body or blood after the substance (e.g., a drug) has been discontinued. The knowledge of the half-life is, for example, useful for the determination of the frequency of administration of a drug (the number of intakes per day) for obtaining the desired blood concentration.

The methods of the present invention allow, for example, to determine the t max and the t1/2 by monitoring the fluorescence signal over a period of time. Accordingly, by determining the maximum signal intensity and the lowest signal intensity, t max and t ½ can be determined. It is thus not necessary to draw blood samples over a determined period of time as is usually done to determine t max and t ½.

However, t max and/or t ½ can optionally be determined in the usual way by drawing blood samples. t max and t ½ values may then be compared to the t max and t ½ values as determined by the methods of the present invention. Accordingly, the methods of the present invention allow, for example, improving the dosage of a drug, i.e., the aim is to reach the prescription of each drug at the dosage which ensures the best efficacy and the minimum of adverse effects for a subject.

“Monitoring or determining the blood clearance of a fluorescent analyte” further includes that the amount of a fluorescent analyte cleared from the blood or blood circulation of a subject per time is monitored or determined. For fluorescent analytes hat exhibit substantial plasma protein binding, clearance is generally defined as the total concentration (free+protein-bound) and not the free concentration.

An analyte may be filtered out or cleared by, for example, processing by the kidneys, liver, gut, lung, or cells of the immune system such as professional antigen presenting cells. These data can be obtained in addition to the results obtained by the methods of the present invention (which is further explained herein below).

In other sites than the kidneys, however, where clearance is made by membrane transport proteins rather than filtration, extensive plasma protein binding may increase clearance by keeping concentration of free substance fairly constant throughout the capillary bed, inhibiting a decrease in clearance caused by decreased concentration of free substance through the capillary.

An analyte may also be filtered out or cleared by, for example, target mediated clearance such as binding of an antibody to a tumor or solid tumor.

The term “tumor” as used herein refer to or describe the physiological condition in mammals that is typically characterized by unregulated cell growth. Examples of tumors include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, and melanoma.

The term “solid tumor” when used herein refers to tumors elected from the group of gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer, preferably breast cancer.

At least three different light source-detection technologies exist which can be employed alone or in any combination in the methods of the present invention, depending on the intention of the method.

The simplest is continuous wave (CW) imaging. This technique uses excitation light of constant intensity and measures either (1) the signal due to a distribution of excited fluorophores or (2) the attenuation of light (due to tissue absorption and scattering) employing multiple source-detector pairs. The technique is technically relatively simple and usually offers the best signal-to-noise (SNR) characteristics.

A more elaborate approach is to use intensity modulated (IM) excitation light at a single or at multiple frequencies. With this method, modulated light attenuation and phase shifts, relative to the incident light, can be measured for multiple source-detector pairs. Compared to a CW measurement, which yields intensity attenuation, the IM technique offers two pieces of information, i.e., intensity attenuation and phase shift per source-detector pair. Amplitude and phase are usually uncorrelated measurements and can more efficiently resolve the absorption and scattering coefficient of intrinsic contrast. In the fluorescence mode, the technique can image two sets of information, fluorophore concentration and fluorescence lifetime.

The third approach, the time-resolved (TR) technique, uses short pulses of excitation light injected into the eye and/or the tissue. The technique resolves the distribution of times that the detected photons travel into the medium for multiple source-detector pairs. Time-resolved methods contain the highest information content per source-detector pair, comparable only to the IM method performed simultaneously at multiple frequencies. This can be easily explained when one considers that the Fourier transform of the time-resolved data yields information at multiple frequencies up to 1 GHz, including the continuous wave components (f=0 MHz) used by the previous two methods. Therefore, the time-resolved method offers a CW component for direct comparison with the CW system, but also intensity attenuation and phase-shift measurements at multiple-frequencies (via the Fourier transform) that can image intrinsic absorption and scattering, and also fluorophore concentration and fluorescence lifetime.

In fluorescence imaging, filtered light or a laser with a defined bandwidth, comprising at least one, i.e. one, two, three, four, five, or even more predetermined wavelength(s), is used as a source of excitation light. “Predetermined wavelength” means that the excitation light comprises defined spectral components (including a single wavelength, a single band of wavelengths, more than one wavelength, or more than one band of wavelengths) which are capable of exciting fluorescent light from the respective fluorophore (comprised by the fluorescent entity and/or fluorescent analyte). If more than one predetermined wavelength is employed, it is preferred that these at least two wavelengths are distinguished or distinguishable from another. As used herein, the term “excitation light” is used to describe light generated by an excitation light source. The excitation light includes, but is not limited to, spectral light components (i.e., wavelengths) capable of exciting fluorescence from a fluorophore. The spectral components in the excitation light that are capable of exciting fluorescent light can include a single wavelength, a single band of wavelengths, more than one wavelength, or more than one spectral band of wavelengths. The spectral components in the excitation light that are capable of exciting fluorescent light can include one or more wavelengths in the visible spectral regions of about 400 to 700 nanometres (nm). However, the spectral components in the excitation light that are capable of exciting fluorescent light can also include one or more wavelengths in the other spectral regions, for example, in the near infrared (NIR) spectral region of about 700 to 1000 nanometres, or in the ultra-violet (UV) spectral region of about 1 to 400 nanometres. The excitation light can further include spectral components that do not excite fluorescent light. The spectral components of the excitation light that are capable of exciting fluorescent light can have wavelengths shorter than the fluorescent light that they excite. However, in other arrangements, some additional spectral components of the excitation light can have wavelengths longer than the fluorescent light that they excite. In a preferred embodiment, the excitation light comprises a spectral band in the range of about 671 to 705 nm (ICG).

The excitation light may be continuous in intensity, continued in wave, pulsed, or may be modulated (for example by frequency or amplitude) or any suitable combination thereof. In some embodiments, the excitation light is coherent light, e.g., laser light. In other embodiments, the excitation light is incoherent light, e.g., photons generated from an LED or filtered light generated from black body radiation (e.g. incandescent, halogen, or xenon bulb). In other embodiments, the excitation light is a combination of coherent and incoherent light.

An imaging system useful in the practice of this invention preferably includes three basic components: (1) excitation light, (2) a means for separating or distinguishing excitation light and emission light (preferably a software and/or hardware filter(s) which might be fitted to the excitation light and/or to the detection system), and (3) a detection system for receiving the light emitted from at least one fluorescent label and/or from the fluorescent entity and/or from the fluorescent analyte of the invention (optical detector). It is envisaged that the light source (excitation means) may optionally (i) comprise a pre-determined or tunable filter. The light source can be a suitably filtered white light, i.e., bandpass light from a broadband source. For example, light from a 150-watt halogen lamp can be passed through a suitable bandpass filter. In some embodiments, the light source is a laser. See, e.g., Boas et al., 1994, Proc. Natl. Acad. Sci. USA 91:4887-4891; Ntziachristos et al., 2000, Proc. Natl. Acad. Sci. USA 97:2767-2772; Alexander, 1991, J. Clin. Laser Med. Surg. 9:416-418. Information on near infrared lasers for imaging can be found at http://www.imds.com and various other well-known sources. A high pass or bandpass filter (e.g. 700 nm) can be used to separate optical emissions (emission light) from excitation light. Any suitable light detection/image recording component (an optical detector), e.g., charge coupled device (CCD) systems, a photodiode, a photoconductive cell, a complementary metal oxide semiconductor (CMOS) or photomultiplier tubes can be used in the invention. Said components are explained in more detail herein below. The choice of light detection/image recording will depend on factors including type of light gathering/image forming component being used. Selecting suitable components, assembling them into an imaging system of the invention, and operating the system is within ordinary skill in the art.

The excitation light travels from the cornea to the retina of the eye, whereby it passes the pupil. When the excitation light encounters a fluorescent label, the light is absorbed. Fluorescence occurs when the fluorescent label relaxes to its ground state after being excited. The fluorescent label then emits light that has detectably different (distinguishable) properties i.e., spectral properties—e.g. a slightly longer wavelength etc., from the excitation light. A part of the absorbed energy is transformed into heat. This loss of energy causes a wavelength shift from the shorter excitation wavelength to a longer emission wavelength. This process is known as the Stokes-Shift. However different optical phenomena like those described in Xu et al. (1996), Proc. Natl. Acad. Sci. 93: 10763-10768 can also be used to generate fluorescence.

The excitation light, which is directed onto a delineated region comprising at least a portion of the pupil of the subject, may travel along the optical axis of the respective eye, or not, or parts of the excitation light travel along the optical axis of the respective eye, whereas other parts do not. It is also envisaged that, for example in case of multiple light sources leading to multiple excitation lights, which are preferably distinguishable, parts of the excitation light travel along the optical axis of the respective eye, whereas other parts do not. The “optical axis” of an eye is a well known term and which is defined as the imaginary line drawn through the center of the eye perpendicular to its anterior and posterior surfaces, or defined as the longest sagittal distance between the front or vertex of the cornea and the furthest posterior part of the eyeball (both definitions are well accepted in the art).

In the context of the present invention, it is envisaged that excitation light comprising at least one, i.e. one, two, three, four, five or even more predetermined and preferably distinguished or distinguishable wavelength(s), is directed onto a delineated region of the subject, said delineated region comprising at least a portion of the pupil of the subject. A “delineated region comprising at least a portion of the pupil of the subject” thereby encompasses, at most (maximal), the whole body of the subject or any smaller part of that body, provided that the said smaller part still encompasses at least a portion of the pupil of the subject (for example the head, or the head and the shoulders, or the head and the upper part of the body). Said delineated region may be smaller than the eye of the subject or larger than the eye of the subject. It is preferred that said delineated region comprises the entire pupil or even the whole eye (i.e. the eyeball) of the subject, the whole eye being more preferred. “Whole eye” specifically includes the visible part of the eye (visible from outside).

“Eye”, “eyeball” or “whole eye” are used interchangeably. “The eye” includes one eye of the subject or both eyes of the subject.

An example of an imaging system is the MAESTRO system, which is exemplified in FIG. 3. It is a near-infra red fluorescence imaging system. The MAESTRO system is a preferred imaging system that may be applied in connection with the embodiments of the present invention.

The MAESTRO system is a planar fluorescence-reflecting-imaging system that allows a noninvasive in vivo fluorescence measurement. In this multispectral analysis, a series of images are captured, at specific wavelengths. The range of wavelengths captured should cover the expected spectral emission range of the label present in the specimen. The result will be a series of images called “image cube” and it is the data within this series of images that is used to define the individual spectra of both auto-fluorescence and specific labels. Many labels of biological interest have emission spectra that are so similar that separation using expensive narrow band filters is difficult or impossible. A single long pass emission filter replaces a large collection of emission filters. In addition to the natural auto-fluorescence of the skin, fur, sebaceous glands, there is also distinct auto-fluorescence from commensal organisms (fungi, mites, etc.) and ingested food (chlorophyll). Multispectral analysis is able to separate all of these signals from the specific label applied to the specimen through the mathematically disentanglement of the linear signal mixture (unmixing) of the emitted fluorescent lights as long as the emission spectrum of the desired signal and of the auto-fluorescence are known.

Measurement with the MAESTRO system works as follows: The illumination module is equipped with a xenon lamp (Cermax) that excites white light. Through a downstream connected excitation filter (chosen by the experimenter), the light is delimitated to a, for the experiment, desired wavelength range and conducted via an optical fiber into the imaging module. In here, the restricted light is partitioned into four optical fibers that illuminate the anesthetized test animal. The MAESTRO system chooses the optimal exposure time automatically, so that there is no risk of overexposure. The emitted fluorescence light of the activated fluorescent probe is selected with an emission filter (see Table 1) and conducted through a liquid crystal (LC) to a high sensitive, cooled CCD-camera. The liquid crystal enables the camera a selective picture recording of a specific wavelength. The wavelength measurement range depends on the selected filter set (blue, green, yellow, red, deep red, NIR) and pictures are recorded in steps of 10 nm. The spectral information of each single picture is combined in one “picture package” that is called “image cube”.

TABLE 1
Maestro filter sets.

| Maestro | | Excitation | | Acquisition |
|---|---|---|---|---|
| Filter Set | Part # | Filter | Emission Filter | Settings* |
| Blue | M-MSI-FLTR-BLUE | 445 to 490 nmm | 515 nm | 500 to 720 in |
| | | | longpass | 10 nm steps |
| Green | M-MSI-FLTR-GREEN | 503 to 555 nm | 580 nm | 550 to 800 in |
| | | | longpass | 10 nm steps |
| Yellow | M-MSI-FLTR- | 575 to 605 nm | 645 nm | 630 to 850 in |
| | YELLOW | | longpass | 10 nm steps |
| Red | M-MSI-FLTR-RED | 615 to 665 nm | 700 nm | 680 to 950 in |
| | | | longpass | 10 nm steps |
| Deep Red | M-MSI-FLTR-DEEP- | 671 to 705 nm | 750 nm | 730 to 950 in |
| | RED | | longpass | 10 nm steps |
| NIR | M-MSI-FLTR-NIR | 710 to 760 nm | 800 nm | 780 to 950 in |
| | | | longpass | 10 nm steps |

The analysis with the MAESTRO system works as follows:

Each recording compose of 12 bit black-and-white pictures that can be illustrated in 4096 different gray scales and therefore it is possible to discriminate between smallest differences in emission intensities. In contrast, the human eye is able to distinguish between 30-35 grey scales. Those values for the emission intensities (grey scales) are plotted against the wavelength range and as a result, we obtain the emission spectra of each probe and the tissue auto-fluorescence. The software subdivides the three fundamental colours (red, green, blue) to the wavelength range used for the imaging cube whereby the black-and-white pictures turn into coloured image. Out of these acquired multispectral information the system is able to differentiate between injected probes and auto-fluorescence of any source. The program is using a spectral library, where the single spectra of each pure probe and the spectra acquired by imaging the study animals (for example Balbc/nude or Scid Beige mice) without any injection (mouse auto-fluorescence). By knowing the exact spectra of the pure imaging and of the auto-fluorescence, the system is able to filter the whole image for the desired spectra and assign a colour to each of them. The originated image (unmixed composite image) shows the present spectra in different colours. To visualize the intensity distribution of the probe signal, it is possible to illustrate the signal in false colours, whereas low intensities are, for example, blue and regions of high intensities are, for example, red. Besides that, one can define a detection limit for the signal intensity of the probe, which allows reducing the signal of circulating probes and unspecific bindings.

Comparison and quantification with the MAESTRO system works as follows:

The MAESTRO's ability to compare fluorophore regions of an image makes it easy to compare the fluorescent signal intensities during therapy. The program provides tools for the comparison of different signal intensities in tumor regions (compared images). Since all images are taken at optimal exposure times, they differ depending on the strength of signals. For a reliable comparison, the pictures are standardized to one exposure time, resulting in an illustration of differences in signal intensities. By manually drawing and modifying measurement regions, signal intensities can be quantified in intensity values. Once a measurement area is selected around the tumor, it can be cloned and moved to the next image to be compared with. Each region is calculated in pixels and mm2 based on the current settings (stage height and binning). As a result, it gives information about the average signal, total signal, max. signal and average signal/exposure time (1/ms) within the created measurement area.

Another imaging technique which may be applied in connection with the present invention is the FMT technology (fluorescence molecular tomography), a laser based three-dimensional imaging system, which provides non-invasive, whole body, deep tissue imaging in small animal models and generates 3D reconstruction of fluorescence sources and/or allows measurement of fluorescence of fluorescence labelled analytes. The FMT technology is described, for example, in U.S. Pat. No. 6,615,063.

A further imaging technique which may be applied in connection with the present invention is the optical imaging method described in WO 2007/143141. This imaging technique for producing an image of a subject including a delineated region comprises: acquiring a time series of image data sets of a targeted optical contrast substance (fluorescent dye or label, a luminescent dye or an absorbing dye) within the subject using an optical detector, wherein each image data set is obtained at a selected time and has the same plurality of pixels, with each pixel having an associated value, analyzing the image data sets to identify a plurality of distinctive time courses, determining the image data sets to identify a plurality of distinctive time courses, determining a respective pixel set from the plurality of pixels which corresponds to each of the time courses, and associating each pixel set with an identified structure, and generating an image of the subject wherein a targeted region is delineated using the identified structures.

In a further embodiment of the methods of the present invention, steps (a) and/or (b) of the methods described hereinabove further include the step of determining the location of the pupil of the eye. The location of the pupil can be determined previous to, during and/or after step (a) and/or (b) of the methods of the invention. Means and methods which are necessary to determine the location of the pupil of the eye are well known to the skilled person, and can be exemplified by the pupillometers described in U.S. Pat. No. 5,784,145 or U.S. Pat. No. 6,820,979.

It is also envisaged that the methods of the present invention, further comprise:
(i) determining the area of said portion of the pupil in step (a), and/or
(ii) determining the area of the pupil of said eye in step (b).

It will be understood that it might be wanted or might happen that different, i.e. not identical delineated regions are employed in step (a) and (b), which will result in a situation where for example a larger area is excited whereas a smaller area is used for receiving the emitted light (or vice versa). It might therefore be wanted/necessary to determine the area of said portion of the retina, pupil and/or of the eye in order to be able to determine/evaluate the intensity of excitation light and/or emitted light per area. This may be done in order to adjust the signals. Means and methods which are necessary to determine the area of the pupil are well known to the skilled person, e.g. by way of modifying a standard pupillometer, such as that described in U.S. Pat. No. 5,784,145 or U.S. Pat. No. 6,820,979.

It is also envisaged that a delineated region of both eyes of the subject is excited with distinguishable and/or identical excitation lights wherein both eyes are excited simultaneously or consecutively. The invention thus also features methods for selectively detecting, quantifying, monitoring etc. at least two different fluorescent analytes, fluorescent labels or fluorescent entities simultaneously or consecutively, wherein one signal is determined through the one eye and the other signal is determined through the other eye of the subject. Alternatively or additionally, it is also envisaged that at least two fluorescent analytes which are distinguishable from each other are determined/monitored/quantified etc. through one and the same eye of the subject simultaneously or consecutively. To this end, the “different” fluorescent characteristics of the at least two fluorescent analytes may be “unmixed” subsequently, e.g. by way of software aided evaluations. Means and methods to unmix the emission of more than one different fluorophore are well known to the skilled person.

Since the present invention relates to methods for determining, quantifying etc. the presence, pharmacokinetic etc. of a fluorescent analyte in the blood of a subject, it will be understood that the excitation light has to be directed such, that it reaches at least a delineated region of the retina of the eye of the subject.

The light emitted from at least one fluorescent label and/or from the fluorescent entity and/or from the fluorescent analyte of the invention is received through the eye of the subject. “Through the eye” means that the detection system receives the emitted light from the at least one fluorescent label, entity or analyte which travels through the blood stream/blood circulation system of the retina of the eye of the subject. “Through the eye” therefore includes that the emitted light is at least received from a delineated region of the retina (in particular from a delineated region of the blood circulation system of the retina), pupil and/or eye. A “delineated region” therefore encompasses, at most (maximal), the whole body of the subject, or any smaller part of that body, provided that the said part still encompasses at least a portion of the retina (including the blood circulation system therein) of the subject, for example the head, or the head and the shoulders, or the head and the upper part of the body. It is envisaged that said delineated region comprises the pupil, pupil and iris, or even the whole eye (eyeball) of the subject, the whole eye being preferred. Alternatively, said delineated region is smaller than the eye of the subject or larger than the eye of the subject. “The eye” as used in the context of the present invention, includes one eye of the subject or both eyes of the subject.

Said delineated region can be obtained by way of adjusting the optical detector, i.e. adjusting the hardware so as to receive light from the delineated region, and/or by way of a “software filter” which evaluates the delineated region.

It is also envisaged that in the methods of the invention, (a) said excitation light of at least one predetermined wavelength is exclusively directed onto a delineated region comprising at least a portion of the pupil of said subject. “Exclusively” means in this regard that said excitation light is at most (maximal) directed onto one or both eye(s) of the subject (or smaller parts of the eye), but not on any other part of the subject.

It is also envisaged that in the methods of the invention said light which is emitted from said fluorescent analyte with a wavelength distinguishable from the predetermined wavelength of (a), is exclusively received through the eye of said subject. “Exclusively”, in this regard, specifically excludes to receive (for example by way of adjusting the hardware) and/or to evaluate (for example by way of a software filter) any emission light from any other region of the subject, besides the eye, in order to determine the presence of said fluorescent analyte in the blood, quantifying the blood level of the fluorescent analyte or monitoring or determining the blood clearance of said fluorescent analyte. “Eye” thereby includes any part of the eye including the pupil, pupil and iris, or the whole eye of the subject, the whole eye being preferred. “Whole eye” also includes the visible part of the eye of a subject (visible from outside). It is thus envisaged that no emission light from any other region of the subject, besides the eye, is determined.

In a preferred embodiment of the methods of the present invention, (i) said excitation light is directed onto a delineated region of the subject, said delineated region being smaller than the whole body (but still encompassing at least a portion of the pupil of the subject) and being larger than the whole eye of the subject. In this regard, it is also preferred that said light emitted from the fluorescent analyte is exclusively received through the eye, preferably the eyeball, of said subject.

In another preferred embodiment of the methods of the present invention, said excitation light is directed onto a delineated region of the subject, and said light emitted from the fluorescent analyte is exclusively received through the eye of said subject.

It will be understood that the emission light, which is received through the eye of the subject, either travels along the optical axis of the respective eye, or not, or parts of the emission light travel along the optical axis of the respective eye, whereas other parts do not. The “optical axis” of an eye is defined herein elsewhere.

The methods of the present invention further encompass embodiments wherein the optical axis of the excitation light(s) is fully identical with, is not identical with, or only at part identical with the optical axis of the emission light.

As it can be seen in the appended examples, it is now possible to determine non-invasively the presence, amount, half-life, kinetic of a fluorescent analyte of interest in the blood and/or the blood circulation of a subject, simply by way of determining the fluorescent signal (emission) which is received from one and/or both eye(s) of said subject. In other words, although it was known in the art that the fluorescent compound which is administered to a subject results, inter alia, in a signal received from the eye of said subject, it remained hidden that the very signal precisely reflects the situation which occurs in the blood (blood circulation) of said subject (either qualitatively, quantitatively and over the time). Thus, the emitted light which is received through the eye of a subject, actually does not resemble the distribution of a fluorescent analyte in the eye, but, instead reflects the distribution of said analyte in the blood or the blood circulation. The correlation of the signal obtained through the eye of the subject with the actual situation (concentration, presence etc.) of said analyte in the blood is neither disclosed nor suggested in the art. Thus, only by that knowledge, it is now possible to reliably detect and exclude the presence of a fluorescent analyte in the blood of a subject. It is for example envisaged to administer a fluorescent analyte at a point of time and to detect its presence some time later (for example after a predetermined period of time)—provided that the signal is not detectable, it is now possible to conclude that the analyte is no longer present in the blood of the respective subject. Analogously, it is now much easier to determine the biological half-life of a fluorescent analyte under in vivo conditions, as it is possible to determine the signal intensity of the emission light (and thereby the theoretical amount of said analyte) within very short time intervals thereby allowing to indicate the course of degradation and/or secretion and/or clearance of the fluorescent analyte from the blood of the respective subject with a significantly increased precision. This measurement is non-invasive and therefore almost stress free for the subject, who is for example a non-human test animal, thereby allowing minimizing the time intervals between two measurements. One may wish also to evaluate the secretion pathway of the fluorescent analyte and/or the organ distribution of said analyte simultaneously. Further, the present invention provides for a screening system, preferably in non-human test animals, by using at least two fluorescent analytes characterized by different second entities (for example different drugs or interaction partners, i.e. two or more binding partners which specifically interact with each other such as, antigen—antibody, antibody—antibody, multimeric protein complexes, protein—protein binding, lectin—sugar binding etc.) and labeled with distinguishable fluorescent labels (which differ e.g. in their emission spectra, and/or are suitable for the evaluation of FRET effects), in order to evaluate the in vivo interaction between these two second entities as such (thereby evaluating whether two entities interact in vivo or not), or to screen for substances which either increase (agonists) or decrease (antagonists) said interaction.

The proposed non-invasive approach thus allows to determine for example serum peak levels (tmax), serum half life time (t1/2) and/or c0, by significantly reducing the number of animals, minimizing animal stress, improving quality of data sets, and improving analytical efficacy with regard to time and costs. Advantageously, the methods of the invention can be combined with in vivo imaging methods known in the art (e.g. whole body in vivo imaging in order to detect the (optionally time-dependent) organ distribution of a fluorescent analyte which will gain further insight into the pharmacological profile of a compound of interest. It is also envisaged that by way of the methods of the present invention, it is now possible to detect the presence of agents which travel with/through the blood stream, for example determining the presence of a pathogenic agent in a non-invasive fashion in vivo, optionally in real-time or over the time. To this end, it is envisaged to administer a fluorescent analyte, label or entity comprising an epitope binding domain which is specific for a target, for example a pathogenic agent, and to determine the presence of said target in the blood by way of receiving the emitted light through the eye(s) of the respective subject. Activated or activatable fluorescent labels/entities are preferred in this regard. Alternatively, it is also envisaged to administer a fluorescent agent comprising an epitope binding domain which is specific for a target, for example a pathogenic agent, and to determine the presence of said target against the presence (or administration) of a test-compound (for example a drug) which is known to exerts or which is expected to exert an effect on that target effect (for example an antibiotic provided that the target is a pathogenic bacterial cell) and to determine said effect by way of receiving the emitted light through the eye(s) of the respective subject. Provided that the test compound is effective, the target is for example eliminated from the blood stream which is indicated by way of the absence of the fluorescent signal received through the eye of said subject. The methods of the present invention, therefore, may be used for the screening of therapeutic agents, optimization of therapeutic agents, determination of the pharmacokinetic profiles of therapeutic agent, screening of formulations, determination of suitable ways of administration (i.v., i.p etc.) and so forth.

The methods of the present invention are non-invasive. “Non-invasive” as used herein means that the methods, uses and/or devices of the invention do not create skin breaks, particularly breaks of the cornea or sclera of the eye of a subject, but do allow and involve contact of the eye, including its cornea or sclera, with radiation and, likewise, penetration of the eye, including its cornea or sclera, by radiation. Radiation thereby includes all kinds of light such as e.g. excitation light, emission light etc. which is described herein in the context of the present invention. The “cornea” of the eye of a subject is the transparent front part of the eye that covers the iris, pupil, and anterior chamber. The “sclera” is the opaque, fibrous, protective, outer layer of the eye containing collagen and elastic fibers, which forms the posterior five sixths of the connective tissue coat of the globe. It is continuous with the cornea.

The “pupil” is a circular opening located in the center of the iris of the eye that controls the amount of light (for example the excitation light) that enters the eye. The iris is a contractile structure, consisting mainly of smooth muscle, surrounding the pupil. Light such as the excitation light, enters the eye through the pupil, and the iris regulates the amount of light by controlling the size of the pupil. In optical terms, the anatomical pupil is the eye's aperture and the iris is the aperture stop. The image of the pupil as seen from outside of the eye is the entrance pupil. In an optical system, the entrance pupil is a virtual aperture that defines the area at the entrance of the system that can accept light. In the context of the present invention, “pupil” or “entrance pupil” may be used interchangeably.

The extraordinary progress of imaging methods as provided by the present invention, allows the visualization of the performance of any analyte in the blood stream, for example that of drugs and drug delivery systems under in vivo conditions. Detailed and quantitative information about the location and concentration of the drug and carrier can be obtained as a function of time, thereby enabling a more profound understanding of biological effects. This information is crucial to the design of optimized drug and/or drug delivery systems. In general, the technology presented can be used (a) optimize a given drug, for example by way of (chemical) modification of the drug (b) to evaluate/optimize new designed formulations for a desired second entity or fluorescent analyte; (c) to evaluate/optimize the dosage of a second entity or fluorescent analyte; (d) to evaluate/optimize different routes of administration for a desired second entity or fluorescent analyte, for example systemic or local, intradermal, intramuscular, intraperitoneal, intravenous, subcutaneous, intranasal, dermal, epidural, oral, intraventricular and intrathecal injection, or pulmonary administration e.g., by use of an inhaler or nebulizer. By using different second entities (for example different drugs) labeled with fluorescent labels which differ in their emission spectra, interaction studies between these two second entities (for example drug-drug) can be performed. In addition, pharmacokinetic data can be generated with this new approach in the disease areas and/or in genetically engineered preclinical models (e.g. FcRn knock-outs).

The non-invasive methods as described herein are, thus, able to determine, quantify or monitor the presence, amount, kinetic (e.g. the plasma clearance) of a fluorescent analyte in blood in real-time and/or over a period of time. Said period of time depends on the intention of the experiment/method, i.e. it might be wanted to analyze the blood concentration (exemplified by a concentration-time profile) of an antibody which might take up to a period of several days or even weeks, or one might want to analyze the half life of a fast-degradable analyte, which might occur within several minutes or even within several seconds. The biological half-life or elimination half life of a substance is the time it takes for a substance (drug, radioactive nuclide, or other) to lose half of its pharmacologic, physiologic, or radiologic activity. By way of the methods of the present invention, it is now possible to monitor and determine the blood concentration (for example the plasma clearance) of any wanted fluorescent analyte over the time, in vivo, in real-time, without a need to take blood samples (i.e. in a non-invasive fashion). Over the time includes but is not limited to time intervals of one, two, three, four, five or even more months, days, hours, minutes or seconds. The time intervals may comprise one or more breaks which are for example necessary to feed the subject, to renew narcotic treatments (provided that they are wanted), to moisten the eyeball etc.

“Blood” means whole blood including plasma and the cellular component of blood. “Plasma” or “plasma of a subject” as used herein means the liquid component of blood in which the blood cells in whole blood would normally be suspended. It follows that in the context of the methods of the present invention, the presence, blood level, and/or blood clearance of the fluorescent analyte is determined in whole blood. Therein, the said fluorescent analyte may be free floating (unbound) and/or may be bound. “Bound” includes that the fluorescent analyte is for example bound to and/or bound by, the cellular components of the whole blood, pathogenic agents, antibodies and/or functional fragments thereof, proteins (for example to proteins within blood plasma like human serum albumin, lipoprotein, glycoprotein, α, β, and γ globulins), peptides, enzymes, toxins, vitamins, hormones, cytokines, therapeutic agents/compounds (drugs), nucleic acids, receptors, receptor ligands, cellular targets such as tumor cells, (micro)metastases or circulating tumor cells (CTCs) which travel through the blood, tumor-antigens, tumor-markers like β-HCG, CA 15-3, CA 19-9, CA 72-4, CFA, MUC-1, MAGE, p53, ETA, CA-125, CEA, AFP, PSA, PSMA etc, drugs, or any other kind of substance which is (a) present in the blood and (b) binds to and/or is bound by the fluorescent analyte of the present invention by way of any suitable binding reaction like, for example, antigen-antibody binding; receptor-ligand binding, binding based on nucleic acid hybridization, lectin-sugar binding, protein-protein binding, protein-nucleic acid-binding etc. The “cellular component of blood” includes the blood cells, including red blood cells (erythrocytes), white blood cells (such as leukocytes) and platelets. “Cellular targets” which may be present in addition to the cellular component of blood includes any other cell type which is known to be or suspected to be present in whole blood, for example tumor cells, and/or metastases.

The fluorescent analyte of the present invention comprises at least two different entities, namely a fluorescent entity and a second entity. It is, however, also contemplated that the fluorescent analyte comprises further entities, for example protection groups to enhance the plasma half-life and/or further non-fluorescent labels such as chemiluminescent or radioactive labels.

It is also envisaged that the fluorescent analyte of the present invention is employed as a mixture comprising in essence (a) the fluorescent analyte (which comprises, for example, the second entity X coupled to the fluorescent entity) and (b) said second entity X without any fluorescent entity/label. The apportionment between the non-labeled second entity X and the labeled second entity X (i.e. the fluorescent analyte comprising X) is variable and includes ratios of 1:10, 1:5, 1:1, 2:1, 5:1, 10:1, 100:1, 1000:1 etc. It is preferred that the above mentioned mixtures comprise equal to or less than 10%, 9%, 8%, 7%, 6%, 5%, 4%, 3%, 2%, 1%, 0.5%, 0.1%, 0.01%, 0.001% etc. of fluorescently labeled second entity X (fluorescent analyte) when compared to the total amount of said second entity X in the mixture. Such mixtures are described in WO 2008/119493.

The “fluorescent entity” is or comprises at least one fluorescent label which allows for the detection of the fluorescent analyte of the invention by way of the methods/uses/devices as disclosed herein. It will be understood that the fluorescent entity of the fluorescent analyte is the collectivity of fluorescent labels which are directly and/or indirectly attached to the second entity.

The fluorescent entity may comprises the at least one fluorescent label(s) or it may comprise a spacer to which the at least one fluorescent label(s) may be coupled. Said spacer can be exemplified by a microspheres, e.g. a latex bead, a peptide, oligonucleotide, polymeric backbones, or other moiety, e.g., a synthetic moiety, containing degradable bonds to which the at least one fluorescent label and, if applicable, quenchers are covalently linked. The polymeric backbone can be any biocompatible polymer. For example, it can be a polypeptide, a polysaccharide, a nucleic acid, or a synthetic polymer. Polypeptides useful as a backbone include, for example, polylysine, albumins, and antibodies. Poly(L-lysine) is a preferred polypeptide backbone. The backbone also can be a synthetic polymer such as polyglycolic acid, polylactic acid, poly(glycolic-co-lactic) acid, polydioxanone, polyvalerolactone, poly-ε-caprolactone, poly(3-hydroxybutyrate, poly(3-hydroxyvalerate) polytartronic acid, and poly(β-malonic acid). Polymeric backbone design will depend on considerations such as biocompatibility (e.g., toxicity and immunogenicity), serum half-life, useful functional groups (e.g., for conjugating spacers, and protective groups), and cost. Useful types of polymeric backbones include polypeptides (polyamino acids), polyethyleneamines, polysaccharides, aminated polysaccharides, aminated oligosaccharides, polyamidoamines, polyacrylic acids and polyalcohols. In some embodiments the backbone includes a polypeptide formed from L-amino acids, D-amino acids, or a combination thereof. Such a polypeptide can be, e.g., a polypeptide identical or similar to a naturally occurring protein such as albumin, a homopolymer such as polylysine, or a copolymer such as a D-tyr-D-lys copolymer. When lysine residues are present in the polymeric backbone, the e-amino groups on the side chains of the lysine residues can serve as convenient reactive groups for covalent linkage to the spacers. When the polymeric backbone is a polypeptide, preferably the molecular weight of the probe is from 2 kD to 1000 kD. More preferably, its molecular weight is from 4 kd to 500 kd.

The fluorescent entity (as well as the fluorescent analyte) can include one or more protective chains covalently linked to the spacer, e.g. to the polymeric backbone. Suitable protective chains include polyethylene glycol, methoxypolyethylene glycol, methoxypolypropylene glycol, copolymers of polyethylene glycol and methoxypolypropylene glycol, dextran, and polylactic-polyglycolic acid.

A “fluorescent label” as used herein characterizes a molecule which comprises a fluorophore. A fluorophore, which is sometimes also termed fluorochrome, is a functional group in a molecule which will absorb energy of a specific wavelength and re-emit energy at a different wavelength. Said different wavelength, when compared to the said specific (predetermined) wavelength, is re-emitted with a wavelength which is distinguishable from the specific (predetermined) wavelength, for example it is re-emitted with a longer wavelength or with a shorter wave-length, however in the latter case with decreased intensity. The amount and wavelength of the emitted energy depends on both the fluorophore and the chemical environment of the fluorophore.

The principle that a wavelength is re-emitted with a shorter wave-length is applied in multiphoton fluorescence excitation; see Xu et al. (1996), Proc. Nathl. Acad. Sci. 93, 10763-10768. Multiphoton fluorescence excitation can be used in the context of the present invention. For example, a sample is illuminated with a wavelength around twice the wavelength of the absorption peak of the fluorophore being used. For example, in the case of fluorescein which has an absorption peak around 500 nm, 1000 nm excitation could be used. Essentially no excitation of the fluorophore will occur at this wavelength. However, if a high peak-power, pulsed laser is used (so that the mean power levels are moderate and do not damage the specimen), two-photon events will occur at the point of focus. At this point the photon density is sufficiently high that two photons can be absorbed by the fluorophore essentially simultaneously. This is equivalent to a single photon with an energy equal to the sum of the two that are absorbed. In this way, fluorophore excitation will only occur at the point of focus (where it is needed) thereby eliminating excitation of out-of-focus fluorophore and achieving optical sectioning

Three-photon excitation can also be used in the context of the present invention. In this case three photons are absorbed simultaneously, effectively tripling the excitation energy. Using this technique, UV excited fluorophores may be imaged with IR excitation. Because excitation levels are dependent on the cube of the excitation power, resolution is improved (for the same excitation wavelength) compared to two photon excitation where there is a quadratic power dependence. It is possible to select fluorophores such that multiple labeled samples by can be imaged by combination of 2- and 3 photon excitation, using a single IR excitation source.

Two-photon excitation microscopy can also be used in the context of the present invention. It is a fluorescence imaging technique that allows imaging living tissue up to a depth of one millimeter.

The concept of two-photon excitation is based on the idea that two photons of low energy can excite a fluorophore in a quantum event, resulting in the emission of a fluorescence photon, typically at a higher energy than either of the two excitatory photons. The probability of the near-simultaneous absorption of two photons is extremely low. Therefore a high flux of excitation photons is typically required, usually a femto second laser.

Two-photon absorption is combined with the use of a laser scanner.[4] In two-photon excitation microscopy an infrared laser beam is focused through an objective lens. The Ti-sapphire laser normally used has a pulse width of approximately 100 femto seconds and a repetition rate of about 80 MHz, allowing the high photon density and flux required for two photons absorption and is tunable across a wide range of wavelengths.

The most commonly used fluorophores have excitation spectra in the 400-500 nm range, whereas the laser used to excite the fluorophores lies in the ˜700-1000 nm (infrared) range. If the fluorophore absorbs two infrared photons simultaneously, it will absorb enough energy to be raised into the excited state. The fluorophore will then emit a single photon with a wavelength that depends on the type of fluorophore used (typically in the visible spectrum). Because two photons need to be absorbed to excite a fluorophore, the probability for fluorescent emission from the fluorophores increases quadratically with the excitation intensity. Therefore, much more two-photon fluorescence is generated where the laser beam is tightly focused than where it is more diffuse.

It is envisaged that a fluorescent entity of the present invention comprises at least one, i.e. one, two, three, four, five, six, seven, eight, nine, ten, 15, 20, 30, 40, 50, 60, 70, 80, 90, 100 or even more fluorescent labels. These fluorescent labels may be identical or different, i.e. it is envisaged that the fluorescent entity as used in the context of the present invention comprises just one sort of fluorescent labels or a mixture of at least two, three, four, five or even more different sorts of fluorescent labels. “Just one sort” means that the fluorescent labels contain one and the same fluorophore, while different sorts means that the different fluorescent labels comprise different fluorophores and therefore show different absorptions and/or emission characteristics. These “different” characteristics may be “unmixed” subsequently, e.g. by way of software aided evaluations. Means and methods to unmix the emission of more than one different fluorophore are well known to the skilled person.

The fluorescent label can be covalently and/or non-covalently linked to the spacer or to the second entity of the fluorescent analyte, using any suitable reactive group on the fluorescent label and a compatible functional group on the spacer or the second entity.

In the context of the present invention, said fluorescent label is preferably selected from the group comprising quantum dot agents, fluorescent proteins, fluorescent dyes, pH-sensitive fluorescent dyes, voltage sensitive fluorescent dyes and/or fluorescent labeled microspheres.

“Quantum dot agents” or “Quantum dots”, also known as nanocrystals, are a special class of materials known as semiconductors, which are crystals composed of periodic groups of II-VI, III-V, or IV-VI materials.

“Fluorescent protein” includes for example green fluorescent protein (GFP), CFP, YFP, BFP either enhanced or not. Further fluorescent proteins are described in Zhang, Nat Rev Mol Cell Biol. 2002, 12, pages 906-18 or in Giepmans, Science. 2006, 312, pages 217-24.

“Fluorescent dyes” includes all kinds of fluorescent labels including but not limited to, Fluorescein including all its derivatives like for example FITC; Rhodamine including all its derivatives such as tetramethylrhodamine (TAMRA) and its isothiocyanate derivative (TRITC), sulforhodamine 101 (and its sulfonyl chloride form Texas Red), Rhodamine Red, and other derivatives of rhodamine which include newer fluorophores such as Alexa 546, Alexa 555, Alexa 633, DyLight 549 and DyLight 633); Alexa Fluors (the Alexa Fluor family of fluorescent dyes is produced by Molecular Probes); DyLight Fluor which is a family of fluorescent dyes are produced by Dyomics, ATTO Dyes, which represent a series of fluorescent labels and dyes manufactured by ATTO-TEC GmbH in Siegen, WO/2007/067978 Japan); LaJolla Blue (Diatron, Miami, Fla.); indocyanine green (ICG) and its analogs (Licha et al., 1996, SPIE 2927:192-198; Ito et al., U.S. Pat. No. 5,968,479); indotricarbocyanine (ITC; WO 98/47538), and chelated lanthanide compounds. Fluorescent lanthanide metals include europium and terbium.

As mentioned, an analyte can also be labelled with a near-infrared (NIR) fluorescence label. NIR fluorescence labels with excitation and emission wavelengths in the near infrared spectrum are used, i.e., 640-1300 nm preferably 640-1200 nm, and more preferably 640-900 nm. Use of this portion of the electromagnetic spectrum maximizes tissue penetration and minimizes absorption by physiologically abundant absorbers such as hemoglobin (<650 nm) and water (>1200 nm). Ideal near infrared fluorochromes for in vivo use exhibit:

(1) narrow spectral characteristics,
(2) high sensitivity (quantum yield),
(3) biocompatibility,
(4) decoupled absorption and excitation spectra, and
(5) photo stability.

Various near infrared (NIR) fluorescence labels are commercially available and can be used to prepare a fluorescent entity according to this invention. Exemplary NIRF labels include the following: Cy5.5, Cy5 and Cy7 (Amersham, Arlington Hts., IL; IRD41 and IRD700 (LI-COR, Lincoln, Nebr.); NIR-I, (Dejindo, Kumamoto, Japan); LaJolla Blue (Diatron, Miami, Fla.); indocyanine green (ICG) and its analogs (Licha, K., et al., SPIE—The International Society for Optical Engineering 1996; Vol. 2927: 192-198; U.S. Pat. No. 5,968,479); indotricarbocyanine (ITC; WO 98/47538); and chelated lanthanide compounds and SF64, 5-29, 5-36 and 5-41 (from WO 2006/072580). Fluorescent lanthanide metals include europium and terbium. Fluorescence properties of lanthanides are described in Lackowicz, J. R., Principles of Fluorescence Spectroscopy, 2nd Ed., Kluwer Academic, New York, (1999).

“Fluorescent microspheres” are described in great detail in WO/2007/067978 which is incorporated herein by reference.

In a further embodiment of the present invention, at least one fluorescent label of the fluorescent entity is activatable. It is also envisaged that the fluorescent entity is activatable.

As mentioned before, it is known that the amount and wavelength of the energy emitted by a fluorescent dye depends on both the fluorophore and the chemical environment of the fluorophore. It follows that fluorescent dyes may react pH-sensitive or voltage sensitive, i.e. they are activatable by such changes in the chemical environment. Further activatable fluorescent labels are described for example in great detail in US 2006/0147378 A1, U.S. Pat. No. 6,592,847, U.S. Pat. No. 6,083,486, WO/2002/056670 or US 2003/0044353 A1, all of which are incorporated herein by reference.

By “activation” of a fluorescent label/entity is meant any change to the label/entity that alters a detectable property, e.g., an optical property, of the label/entity. This includes, but is not limited to, any modification, alteration, or binding (covalent or non-covalent) of the label/entity that results in a detectable difference in properties, e.g., optical properties e.g., changes in the fluorescence signal amplitude (e.g., dequenching and quenching), change in wavelength, fluorescence lifetime, spectral properties, or polarity. Optical properties include wavelengths, for example, in the visible, ultraviolet, near-infrared, and infrared regions of the electromagnetic spectrum. Activation can be, without limitation, by enzymatic cleavage, enzymatic conversion, phosphorylation or dephosphorylation, conformation change due to binding, enzyme-mediated splicing, enzyme-mediated transfer of the fluorophore, hybridization of complementary DNA or RNA, analyte binding such as association with an analyte such as Na+, K+, Ca2+, Cl−, or another analyte, change in hydrophobicity of the probe environment, and chemical modification of the fluorophore. Activation of the optical properties may or may not be accompanied by alterations in other detectable properties, such as (but not limited to) magnetic relaxation and bioluminescence.

In a further embodiment of the present invention, at least one fluorescent label of the fluorescent analyte is activated once the epitope binding domain of said second entity has bound to its target. It is also envisaged that the fluorescent entity is activated once the epitope binding domain of said second entity has bound to its target.

“Activated” includes the activation of activatable fluorescent labels which have been mentioned herein before. It is for example envisaged that the fluorescent analyte of the invention comprises at least one activatable fluorescent label which is activated by way of proteolytic cleavage (e.g. by way of enzymatic cleavage which releases a cleavable scavenger—such systems are described for example in US 2006/0147378 A1, U.S. Pat. No. 6,592,847, U.S. Pat. No. 6,083,486, WO/2002/056670 or US 2003/0044353 A1). “Activated” also includes “FRET-based” effects. Förster resonance energy transfer (abbreviated FRET), also known as fluorescence resonance energy transfer, resonance energy transfer (RET) or electronic energy transfer (EET), is a mechanism describing energy transfer between two fluorophores. FRET provides an indication of proximity between donor and acceptor fluorophores. When a donor is excited with incident radiation at a defined frequency, some of the energy that the donor would normally emit as fluorescence is transferred to the acceptor, when the acceptor is in sufficiently close proximity to the donor (typically, within about 50 Angstroms for most donor fluorophores). At least some of the energy transferred to the acceptor is emitted as radiation at the fluorescence frequency of the acceptor. FRET is further described in various sources, such as “FRET Imaging” (Jares-Erijman, E. A, and Jovin, T. M, Nature Biotechnology, 21(11), (2003), pg 1387-1395), which is incorporated herein by reference for all purposes. Screening systems based on such FRET effects are well known and described for example in WO 2006107864 which is included herein in its entirety.

The fluorescent analytes of the present invention further comprise a second entity. Said second entity is normally the “analyte” as such, i.e. the analyte whose presence, quantity, kinetic, blood clearance etc. is to be determined by way of the methods, uses and devices disclosed herein. To this end, said second entity is linked with a fluorescent entity and the fluorescent analyte is then determined, quantified, monitored etc. by way of the means and methods of the present invention. It will be understood that the second entity of the fluorescent analyte of the present invention is the collectivity of second entities which are directly or indirectly attached to the fluorescent entity. It is, for example, contemplated that a fluorescent analyte of the present invention comprises more than one, i.e. two three, four, five, or even more second entity(ies). Said second entities can be identical and/or different from each other.

It is also envisaged that the fluorescent entity is activated once it has bound to the second entity (for example based on the above described FRET-effects). Said activation can take place in vitro, and, alternatively, said activation can take place in vivo, i.e. methods are envisaged, wherein said fluorescent entity is to be activated in said subject. “Is to be activated” can occur in a passive fashion, which means that the fluorescent analyte which is characterized by an activatable fluorescent label is administered to the subject and its detectable properties are changed in the subject (e.g. by way of FRET-effects or by way of proteolytic effects); or they can occur in an active fashion, for example by way of administering a protease which activates the fluorescent label/entity in vivo.

It will be understood that the activation of the fluorescent label/entity, preferably, precedes step (b) of the methods of the invention, i.e. precedes the step of receiving the light emitted from said fluorescent analyte. Alternatively, said activation and said receiving occur simultaneously.

It is also envisaged that in the context of the methods of the present invention, said fluorescent analyte, fluorescent entity, fluorescent label, second entity, and/or said target is to be administered to said subject.

It is, alternatively, envisaged that none of the methods of the present invention includes a step of administering said fluorescent analyte, fluorescent entity, fluorescent label, second entity, and/or said target to said subject.

It will be understood that in the context of the methods disclosed herein, said administration of said fluorescent analyte, said fluorescent entity and/or said target precedes step (b) of the methods of the invention, i.e. precedes the step of receiving the light emitted from said fluorescent analyte.

The term “second entity” refers to an analyte whose presence, pharmacokinetic, plasma clearance, biological half-life, peak level etc is to be determined, quantified, monitored etc. by way of the methods, uses and devices of the present invention. The term “second entity” therefore includes, but is not limited to, pathogenic agents, epitope binding domains (either bound or not bound to their target), antibodies and/or functional fragments thereof (either bound or not bound to the target), proteins (for example proteins within blood plasma like human serum albumin, lipoprotein, glycoprotein, α, β, and γ globulins), peptides, enzymes, toxins, vitamins, polysaccharides, lipids, hormones, cytokines, therapeutic agents/compounds (drugs), nucleic acids (for example siRNA), receptors (either bound or not bound to their ligand), receptor ligands (either bound or not bound to their receptor), cellular targets such as tumor cells or (micro)metastases which travel through the blood, tumor-antigens, tumor-markers like β-HCG, CA 15-3, CA 19-9, CA 72-4, CFA, MUC-1, MAGE, p53, ETA, CA-125, CEA, AFP, PSA, PSMA etc, or any other kind of substance whose presence in the blood is of interest. The above terms have been defined herein elsewhere.

It is preferred that the “second entity” exerts a beneficial effect in a medical context, i.e. displays therapeutic and/or diagnostic activity/capabilities, ex vivo and/or in vivo. It follows that in one embodiment of the present invention said second entity comprises a diagnostic and/or therapeutic agent.

“Pathogenic agents” means an agent who causes disease or illness to its host. Pathogenic agents therefore includes all kinds of bacteria like for example species of Escherichia, Salmonella, Shigella, Klebsiella, Vibrio, Pasteurella, Borrelia, Leptospira, Campylobacter, Clostridium, Corynebacterium, Yersinia, Treponema, Rickettsia, Mycoplasma, Coxiella, Neisseria, Listeria, Haemophilus, Helicobacter, Legionella, Pseudomonas, Bordetella, Brucella, Staphylococcus, Streptococcus, Enterococcus, Bacillus, Mycobacterium, Nocardia, etc; viruses like for example viruses of the genus Picornaviridae, Caliciviridae, Reoviridae, Togaviridae, Flaviviridae, Orthomyxoviridae, Paramyxoviridae, Rhabdoviridae, Coronaviridae, Bunyaviridae, Arenaviridae, Rteroviridae, Parvoviridae, Papovaviridae, Adenoviridae, Herpesviridae, Poxyiridae, HAV, HBV, HCV, HIV, HTLV, influenza virus, herpes virus, pox virus, including all known subtypes and variations, HBV, HCV and HIV being preferred; funghi like for example species of Aspergillus, Candida, Cryptococcus, Histoplasma, Blastomyces, Paracoccoides, Mucor, Curvularia, Fusarium etc. including the fungal spores, protozoa or protozoan parasites like for example Entamoeba histolytica or species belonging to the Apicomplexa (particularly the blood borne suborders including Adeleorina, Haemosporida and Eimeriorina, species of the genus Plasmodia being preferred), and/or endoparasites. Pathogenic agents which cause blood-borne diseases are preferred. A “blood-borne disease” is one that can be spread by contamination by blood.

The term “antibody” refers to a monoclonal or a polyclonal antibody (see Harlow and Lane, “Antibodies, A Laboratory Manual”, CSH Press, Cold Spring Harbor, USA, 1988) which binds to a target, or a derivative of said antibody which retains or essentially retains its binding specificity. Preferred derivatives of such antibodies are chimeric antibodies comprising, for example, a mouse or rat variable region and a human constant region. The term “functional fragment” as used herein refers to fragments of the antibodies as specified herein which retain or essentially retain the binding specificity of the antibodies like, separated light and heavy chains, Fab, Fab/c, Fv, Fab′, F(ab′)2. The term “antibody” also comprises bifunctional (bispecific) antibodies and antibody constructs, like single-chain Fvs (scFv) or antibody-fusion proteins. The term “scFv fragment” (single-chain Fv fragment) is well understood in the art and preferred due to its small size and the possibility to produce such fragments recombinantly. Said antibody or antibody binding portion is a human antibody or a humanized antibody. The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A10 239 400 and WO90/07861. The term antibody or functional fragment thereof also includes heavy chain antibodies and the variable domains thereof, which are mentioned in WO 94/04678, WO 96/34103 and WO 97/49805, WO 04/062551, WO 04/041863, WO 04/041865, WO 04/041862 and WO 04/041867; as well as domain antibodies or “dAb's”, which are based on or derived from the heavy chain variable domain (VH) or the light chain variable domain (VL) of traditional 4 chain antibody molecules (see, e.g., Ward et al. 1989 Nature 341, 544-546).

The fluorescent analytes and/or the second entity of the present invention may comprise at least one, i.e. one, two, three, four, five or even more “epitope binding domains”. The term “epitope binding domain” includes, besides the above mentioned antibodies or functional fragments thereof, other binding entities which bind to (specifically bind to) a target such as for example the pathogenic agents, proteins, peptides, enzymes, toxins, vitamins, polysaccharides, lipids, hormones, cytokines, therapeutic agents/compounds (drugs), nucleic acids (for example siRNA), receptors, receptor ligands, cellular targets such as tumor cells or (micro)metastases which travel through the blood, tumor-antigens, tumor-markers, etc.

The terms “target” or “target molecule,” as used herein, refer to any biomolecule of interest to which an epitope binding domain binds. Exemplary targets include, but are not limited to, secreted peptide growth factors, pharmaceutical agents, cell signaling molecules, blood proteins, portions of cell surface receptor molecules, portions of nuclear receptors, steroid molecules, viral proteins, carbohydrates, enzymes, active sites of enzymes, binding sites of enzymes, portions of enzymes, small molecule drugs, cells, bacterial cells, proteins, epitopes of proteins, surfaces of proteins involved in protein-protein interactions, cell surface epitopes, diagnostic proteins, diagnostic markers, plant proteins, peptides involved in protein-protein interactions, and foods, including food ingredients. The target may be associated with a biological state, such as a disease or disorder in a plant or animal as well as the presence of a pathogen. When a target is “associated with” a certain biological state, the presence or absence of the target or the presence of a certain amount of target can identity the biological state.

As used herein, the term “binds” in connection with the interaction between a target and a epitope binding domain indicates that the epitope binding domain associates with (e.g., interacts with or complexes with) the target to a statistically significant degree as compared to association with proteins generally (i.e., non-specific binding). Thus, the term “epitope binding domain” is also understood to refer to a domain that has a statistically significant association or binding with a target.

The term “epitope binding domain” includes, for example, a domain that (specifically) binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin (adnectin); which has been subjected to protein engineering in order to obtain binding to a ligand other than the natural ligand. CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001)

Lipocalins are a family of extracellular proteins which transport small hydrophobic molecules such as steroids, bilins, retinoids and lipids. They have a rigid β-sheet secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1.

Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem. 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomising residues in the first a-helix and a β-turn of each repeat. Their binding interface can be increased by increasing the number of modules (a method of affinity maturation). For further details see J. Mol. Biol. 332, 489-503 (2003), PNAS100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the 13-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796.

Other epitope binding domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Epitope binding domains of the present invention could be derived from any of these alternative protein domains. Examples of further “epitope binding domains” are receptors (specifically binding to their ligand), lectins (specifically binding to polysaccharides), zinc fingers and leucine zippers (binding to nucleic acids), enzymes (specifically binding to their substrate), viruses and bacteria (for example specifically binding to their target cells), nucleic acids (specifically hybridizing to each other) etc.

Therapeutic “epitope binding domains”, and therapeutic antibodies or functional fragments thereof which act as a therapeutic agent (drug) are preferred. Particularily preferred are alemtuzumab, apolizumab, cetuximab, epratuzumab, galiximab, gemtuzumab, ipilimumab, labetuzumab, panitumumab, rituximab, trastuzumab, nimotuzumab, mapatumumab, matuzumab, rhMab ICR62, rhMab B-Ly1 and pertuzumab.

A “therapeutic agent” is an agent wherein the primary purpose of the therapeutic compound is to improve symptoms of a specific disease or adverse medical condition. The term “disease” as used herein, refers to any disordered or incorrectly functioning organ, part, structure, or system of the body resulting from the effect of genetic or developmental errors, infection, poisons, nutritional deficiency or imbalance, toxicity, or unfavorable environmental factors; illness; sickness; or ailment. The term “symptom” as used herein, refers to any phenomenon that arises from and accompanies a particular disease or disorder thereby serving as an indicator. “Therapeutic agent” or “therapeutic compound” includes but is not limited to antibacterial-, antifungal-, antiviral-, antiproliferative-, immunosuppressive-, immunoactivating-, analgesic-, antineoplastic-agents, or histamine receptor antagonists. The term “disease” further includes any impairment of the normal state of the living animal or one of its parts that interrupts or modifies the performance of vital functions that are typically manifested by distinguishing signs and symptoms. For example, a disease may include, but is not limited to, cancer diseases, cardiovascular diseases, neurodegenerative diseases, immunologic diseases, autoimmune diseases, inherited diseases, infectious diseases, bone diseases, and environmental diseases.

The term “antibacterial agent” relates to any compound, which has a growth inhibition or growth restriction activity on bacteria including, e.g. [beta]-lactam antibiotics or quinolone antibiotics. The term further includes an agent selected from the group consisting of nafcillin, oxacillin, penicillin, amoxacillin, ampicillin, cephalosporine, cefotaxime, ceftriaxone, rifampin, minocycline, ciprofloxacin, norfloxacin, erythromycin, tetracycline, gentamicin, a macrolide, a quinolone, a [beta]-lactone, a P-lactamase inhibitor, salicylamide, and vancomycin, sulfanilamide, sulfamethoxazole, sulfacetamide, sulfisoxazole, sulfadiazine, penicillins such as penicillins G and V, methicillin, oxacillin, naficillin, ampicillin, amoxacillin, carbenicillin, ticarcillin, mezlocillin and piperacillin, cephalosporins such as cephalothin, cefaxolin, cephalexin, cefadroxil, cefamandole, cefoxitin, cefaclor, cefuroxine, loracarbef, cefonicid, cefotetan, ceforanide, cefotaxime, cefpodoxime, proxetil, ceftizoxime, cefoperazone, ceftazidime and cefepime, aminoglycosides such as gentamycin, tobramycin, amikacin, netilmicin, neomycin, kanamycin, streptomycin, and the like, tetracyclines such as chlortetracycline, oxytetracycline, demeclocycline, methacycline, doxycycline and minocycline, and macrolides such as erythromycin, clarithromycin, and azithromycin or analogs thereof.

The term “antifungal agent” relates to any compound, which has a growth inhibition or growth restriction activity on fungal species, such as amphotericin, itraconazole, ketoconazole, miconazole, nystatin, clotrimazole, fluconazole, ciclopirox, econazole, naftifine, terbinafine, and griseofulvin.

The term “antiviral agent” relates to any compound that has a growth inhibition or growth restriction activity on viral species, such as aciclovir, famciclovir, ganciclovir, foscarnet, idoxuridine, sorivudine, trifluridine (trifluoropyridine), valacyclovir, cidofovir, didanosine, stavudine, zalcitabine, zidovudine, ribavirin, and rimantatine.

The term “antiproliferative agent” relates to any compound, which inhibits or restricts the cell proliferation, such as methotrexate, azathioprine, fluorouracil, hydroxyurea, 6-thioguanine, cyclophosphamide, mechloroethamine hydrochloride, carmustine, cyclosporine, taxol, tacrolimus, vinblastine, dapsone, nedocromil, cromolyn (cromoglycic acid), and sulfasalazine.

The term “immunosuppressive agent” relates to any compound, which leads to the inhibition or prevention of the activity of the immune system, such as glucocorticoids, cytostatics, drugs acting on immunophilins or TNF-binding proteins. The term also includes cyclophosphamide, anthracycline, mitomycin C, bleomycin, mithramycin, azathioprine, mercaptopurine, methotrexate cyclosporin, an anti IL-2 receptor antibody, an anti-OKT3 antibody and an anti-CD3 antibody, and TNF-α binding monoclonal antibodies such as infliximab (Remicade®), etanercept (Enbrel®), or adalimumab (Humira®).

The term “analgesic agent” relates to any compound used to relieve pain, such as lidocaine, bupivacaine, novocaine, procaine, tetracaine, benzocaine, cocaine, mepivacaine, etidocaine, proparacaine, ropivacaine, and prilocalne.

The term “antineoplastic agent” relates to any compound, which inhibits and combats the development of tumors, such as pentostatin, 6-mercaptopurine, 6-thioguanine, methotrexate, bleomycins, etoposide, teniposide, dactinomycin, daunorubicin, doxorubicin, mitoxantrone, hydroxyurea, 5-fluorouracil, cytarabine, fludarabine, mitomycin, cisplatin, procarbazine, dacarbazine, paclitaxel, docetaxel, colchicine, and vinca alkaloids.

The term “histamine receptor antagonist” relates to any compound, which serves to inhibit the release or action of histamine, such as 2-methylhistamine, 2-pyridylethylamine, 2-thiazolylethylamine, (R)-a-methylhistamine, impromidine, dimaprit, 4(5)-methylhistamine, diphenhydramine, pyrilamine, promethazine, chlorpheniramine, chlorcyclizine, terfenadine, and the like.

The term “toxin” in the context of the present invention relates to any molecule, which is capable of causing disease or cell death on contact or absorption with body tissues by interacting with biological macromolecules such as enzymes or cellular receptors. The term includes but is not limited to botulinum toxins, tetanus toxin, pertussis toxin, heat stable and heat labile E. coli entertoxin, Cholera toxin, Shiga toxin, cytolethal distending toxin, tracheal cytotoxin, diphtheria toxin, clostridial toxins, tetrodotoxin, batrachotoxin, maurotoxin, agitoxin, charybdotoxin, margatoxin, slotoxin, scyllatoxin, calciseptine, taicatoxin, and calcicludine.

The term “hormone” relates to any compound, which carriers as a messenger a signal from one cell (or group of cells) to another via the blood, such as prostaglandine, serotonine, histamine, bradykinin, kallikrein, and gastrointestinal hormones, releasing hormones, pituitary hormones, insulin, vasopressin (ADH), glucagon, enkephalin, calcitonin, and corticosteroids.

The term “vitamin” relates to any compound, which is required as a nutrient in tiny amounts by an organism, such as vitamin A, B1, B2, B3, B5, B6, B7, B9, B12, C, D, E, or K.

The term “receptor-molecules” relates to protein on the cell membrane or within the cytoplasm or cell nucleus that binds to a a ligand and typically transduces a signal, such as metabotropic receptors, G protein-coupled receptors, muscarinic acetylcholine receptors, adenosine receptors, adrenoceptors, GABA receptors, angiotensin receptors, cannabinoid receptors, cholecystokinin receptors, dopamine receptors, glucagon receptors, metabotropic glutamate receptors, histamine receptors, olfactory receptors, opioid receptors, chemokine receptors, calcium-sensing receptor, somatostatin receptors, serotonin receptors, secretin receptors or Fc receptors.

The term “cytokines” relates to soluble proteins and peptides that act as humoral regulators, which, either under normal or pathological conditions, modulate the functional activities of individual cells and tissues and also mediate interactions between cells directly and regulate processes taking place in the extracellular environment. The term encompasses type 1 cytokines produced by Th1 T-helper, type 2 cytokines produced by Th2 T-helper cells, interleukins, chemokines or interferons, e.g. IL-1ra (antagonist), CNTF, LIF, OSM, Epo, G-CSF, GH, PRL, IP10, I309, IFN-alpha, IFN-beta, IFN-gamma, IL2, IL3, IL4, IL5, IL6, IL7, IL8, IL9, IL10, IL11, IL12 (p35+p40), IL13, IL14, IL15, IL16, IL17 A-F, IL18, IL19, IL20, IL21, IL22, IL23 (p19+p40), IL24, IL25, IL26, IL27 (p28-EB13), IL28A, IL28B, IL29, IL30, IL31, IL32, IL33, IL35 (p35-EBI3), LT-alpha, LT-beta, light, TWEAK, APRIL, BAFF, TL1A, GITRL, OX40L, CD40L, FASL, CD27L, CD30L, 4-1BBL, TRAIL, RANK, GM-CSF, M-CSF, SCF, IL1-alpha, IL1-beta, aFGF, bFGF, int-2, KGF, EGF, TGF-alpha, TGF-beta, TNF-alpha, TNF-beta, betacellulin, SCDGF, amphiregulin or HB-EGF, as is known to the person skilled in the art and can be derived, for example, from Tato, C. M. & Cua, D. J. (Cell 132: 900; Cell 132: 500, Cell 132: 324, (2008)) or from Cytokines & Cells Online Pathfinder Encyclopaedia (http://www.copewith-cytokines.de). “Pro-inflammatory cytokines” are also contemplated. The term “pro-inflammatory cytokine” means an immunoregulatory cytokines that favours inflammation. Typically, pro-inflammatory cytokines comprise IL-1-alpha, IL-1-beta, IL-6, and TNF-alpha. These pro-inflammatory cytokines are largely responsible for early responses. Other pro-inflammatory mediators include LIF, IFN-gamma, IFN-alpha, OSM, CNTF, TGF-beta, GM-CSF, TWEAK, IL-11, IL-12, IL-15, IL-17, IL-18, IL-19, IL-20, IL-8, IL-16, IL-22, IL-23, IL-31, and IL-32 (Tato, C. M. & Cua, D. J. Cell 132:900; Cell 132:500, Cell 132, 324 (2008)). These pro-inflammatory cytokines may act as endogenous pyrogens (IL-1, IL-6, TNF-alpha), up-regulate the synthesis of secondary mediators and pro-inflammatory cytokines by both macrophages and mesenchymal cells (including fibroblasts, epithelial and endothelial cells), stimulate the production of acute phase proteins, or attract inflammatory cells. Preferably, the term “pro-inflammatory cytokine” relates to TNF-alpha, IL-15, IFN-gamma, IFN-alpha, IL-1-beta, IL-8, IL-16 and 1 L-22.

The term “nucleic acid” refers to any nucleic acid known to the person skilled in the art, e.g. a polynucleotide like DNA, RNA, single stranded DNA, cDNA, PNA or derivatives thereof. Preferably the term refers to oligonucleotides and polynucleotides formed of DNA and RNA, and analogs thereof, which have selected sequences designed for hybridisation to complementary targets, such as antisense sequences for single- or double-stranded targets, or for expressing nucleic acid transcripts or proteins encoded by the sequences. Analogs include charged and preferably uncharged backbone analogs, such as phosphonates, methyl phosphonates, phosphoramidates, preferably N-3′ or N-5′, thiophosphates, uncharged morpholino-based polymers, and protein nucleic acids (PNAs). Such molecules can be used in a variety of therapeutic regimens, including enzyme replacement therapy, gene therapy, and antisense therapy, for example. Furthermore, the term refers to siRNA, or antisense RNA/DNA. PNAs containing all four natural nucleobases hybridise to complementary oligonucleotides obeying Watson-Crick base-pairing rules, and are a true DNA-mimic in terms of base pair recognition (Egholm et al. Nature 365:566-568 (1993)). The backbone of a PNA is formed by peptide bonds rather than phosphate esters, making it well-suited for anti-sense applications.

The term “protein” includes any polypeptide of interest, including therapeutically active proteins, enzymes, marker proteins etc.

In another embodiment of the methods and uses and devices of the present invention, the fluorescent entity is the fluorescent analyte. This means that the fluorescent entity is the analyte as such, i.e. the fluorescent analyte comprises at least one fluorescent entity but no second entity.

In one embodiment of the present invention, said second entity is directly labeled with said fluorescent entity. The term “directly labeled” includes that the fluorescent entity and the second entity are covalently linked to each other. Said covalent linkage can be built between the fluorescent label(s) and the second entity and/or between the spacer(s), to which the fluorescent label(s) is/are coupled, and the second entity. Provided that the second entity is directly labeled with more than one fluorescent label, it is also envisaged that some (or one) of these fluorescent label(s) are(is) covalently linked to the second entity whereas others are(is) coupled to (a) spacer(s) which is(are) covalently coupled to said second entity. Spacers have been defined herein elsewhere.

Methods for coupling fluorescent labels including NIR fluorescence labels are well known in the art. The conjugation techniques of these labels to an antibody have significantly matured during the past years and an excellent overview is given in Aslam, M., and Dent, A., Bioconjugation (1998) 216-363, London, and in the chapter “Macromolecule conjugation” in Tijssen, P., “Practice and theory of enzyme immunoassays” (1990), Elsevier, Amsterdam.

Appropriate coupling chemistries are known from the above cited literature (Aslam, supra). The fluorescent label, depending on which coupling moiety is present, can be reacted directly with the antibody either in an aqueous or an organic medium. The coupling moiety is a reactive group or activated group which is used for chemically coupling of the fluorochrome label to the antibody. The fluorochrome label can be either directly attached to the antibody or connected to the antibody via a spacer to form a NIR fluorescence label conjugate comprising the antibody and a NIR fluorescence label. The spacer used may be chosen or designed so as to have a suitably long in vivo persistence (half-life) inherently.

In another embodiment of the methods of the present invention, said second entity is indirectly labeled with said fluorescent entity. The term “indirectly labeled” means that the fluorescent entity and the second entity are non-covalently linked with each other. “Non-covalently” includes (a) that the fluorescent entity intercalates into the second entity (such as ethidium bromide which intercalates into nucleic acids); and (b) any kind of suitable binding reaction based on two binding partners which specifically interact with each other in a non-covalent fashion, such as, for example, antigen-antibody binding; receptor-ligand binding, binding based on nucleic acid hybridization, lectin-sugar binding, protein-protein binding, protein-nucleic acid-binding, biotin-streptavidin, DIG—anti DIG antibody, etc. In this context, the fluorescent entity is coupled to one binding partner whereas the second entity is either coupled to the specific counterpart of said binding partner or is said specific counterpart of said binding partner. For example, antibodies or antibody fragments can be produced and coupled to the fluorescent entities or second entity of this invention using conventional antibody technology (see, e.g., Folli et al., 1994, “Antibody-Indocyanin Conjugates for Immunophotodetection of Human Squamous Cell Carcinoma in Nude Mice,” Cancer Res. 54:2643-2649; Neri et al., 1997, “Targeting By Affinity-Matured Recombinant Antibody Fragments of an Angiogenesis Associated Fibronectin Isoform,” Nature Biotechnology 15:1271-1275). Said antibodies or functional fragments thereof may then bind to a specific epitope which might already be present or which is artificially introduced into the “second part” of the fluorescent analyte, i.e. provided that the fluorescent entity is coupled to the antibody or fragment thereof, then the epitope which is specifically bound by the antibody or fragment must be present or must be introduced into the second entity. Or vice versa. Similarly, receptor-binding polypeptides and receptor-binding polysaccharides can be produced and conjugated to fluorescent or second entities of this invention using known techniques.

The indirect labeling can be carried out in vitro or in vivo.

It is also envisaged that said second entity is directly and indirectly labeled at a time. For example one fluorescent label is covalently attached whereas another fluorescent label is coupled to an anti-DIG antibody which recognizes a DIG label on the second entity.

In a further embodiment of the methods of the present invention said fluorescent entity comprises at least one epitope binding domain which is specific for said second entity. In another embodiment of the methods of the present invention said second entity comprises at least one epitope binding domain which is specific for said fluorescent entity. “At least one” includes one, two, three, four, five or even more epitope binding domains which may be of one sort (having the same specificity) or of different sort (having different specificities). In particular, the second entity may comprise at least two different epitope bonding domains, at least one which is specific for the fluorescent entity and at least one which is specific for a target. In a particularly preferred embodiment of the present invention said second entity is coupled to DIG and said fluorescent entity is coupled to an anti-DIG antibody, or vice versa.

It might occur that the fluorescent entity which is present in the fluorescent analyte of the invention alters the desired characteristics of the second entity (for example its pharmacokinetic profile, biological half-life etc.). Accordingly, it might be wanted to compare data which were obtained with the non-labelled second entity by “conventional methods”, e.g. by way of taking blood samples, with data obtained by the methods of the present invention in order to be able to adjust/calibrate the results obtained by the methods of the present invention. It is therefore also envisaged to adapt/calibrate or optimize the methods and/or devices of the present invention. It is furthermore envisaged to adapt/calibrate certain characteristics of the fluorescent analyte (for example its pharmacokinetic characteristics such as stability in the plasma, plasma clearance, target affinity, peak levels in the blood (tmax), plasma half-life etc.). To this end, one could for example select different fluorescent labels, different amounts of label per fluorescent analyte and/or a different fluorescent entity (for example characterized by a different spacer, and/or different amount of fluorophore molecules per molecule spacer) in order to adjust the in vivo characteristics of the fluorescent analyte (e.g. its pharmacokinetic characteristics) to the very characteristics of the non-labelled second entity. It follows that the methods of the present invention may additionally comprise the step of comparing the data obtained by the methods of the invention with data obtained with the non-labelled analyte (e.g. the second entity) in order to (a) adjust the pharmacokinetic characteristics of the fluorescent analyte; and/or (b) to correlate the data obtained with the fluorescent labelled analyte with data obtained with non-labelled analyte (e.g. the second entity); and/or (c) to optimize the fluorescent label (qualitatively and quantitatively, i.e. it may be desired to choose a different fluorophore, and/or to chose a different concentration of fluorophore molecules per molecule of second entity, and/or a different concentration of fluorophore molecules per molecule of spacer of the fluorescent entity). A “non-labelled analyte (e.g. the second entity)” means that said analyte is not fluorescently labelled, i.e. it may be not labelled at all or it may be labelled with other non-fluorescent labels such as radiolabels etc.

“Subject” in the context of the present invention includes any animal which comprises a blood circulation system and at least one eye. An “eye” comprises in this context at least a pupil and a retina, wherein said retina is supplied with blood. Preferably, said subject is a vertebrate, more preferably a mammalian. It is more preferred that said mammalian subject is a non-human animal, a human, a monkey such as cynomolgus, a mouse, a rat, a guinea pig, a rabbit, a horse, a camel, a dog, a cat, a pig, a cow, a goat or a fowl. It is even more preferred that the subject is a mouse, a rat, a rabbit and/or a human.

A non-human subject may represent a model of a particular disease or disorder. It is also envisaged that the subject of the present invention comprises a xenograft, preferably a tumor.

It is also envisaged that the subject of the present invention is a subject to which the fluorescent analyte, fluorescent entity, fluorescent label, second entity, and/or the target is to be administered. Alternatively, the subject of the present invention is a subject who has received the fluorescent analyte, fluorescent entity, fluorescent label, second entity, and/or the target of the invention (which means that the subject is a subject to whom the aforementioned entities have been pre-delivered). “Pre-delivered” includes in this regard, that the entities have been delivered to the subject prior to the methods of the present invention (and all associated embodiments), i.e. before the methods of the invention are to be carried out. It is furthermore envisaged that the subject of the present invention is a subject comprising the fluorescent analyte, fluorescent entity, fluorescent label, second entity, and/or the target of the present invention.

The methods of the present invention can further comprise the step, (c) determining light emitted from said fluorescent analyte with a wavelength distinguishable from the predetermined wavelength of (a), from further regions of the subject.

“From further regions of the subject” means further defined or discrete parts, or regions of the subject, besides the eye or parts of the eye, which might be of interest for any kind of measurement. For example, it is envisaged that the organ distribution and/or accumulation and/or secretion (determination of the secretion pathway) and/or metabolism (for example the generation of metabolites of a drug) of a fluorescent analyte is to be detected and/or evaluated, which will for example aid in the determination of the excretion pathway of said analyte. “Further regions” therefore comprises for example a part of an organ, an organ, blood vessel networks or nervous cell system of the subject. It is also envisaged to correlate the data obtained by the methods of the present invention with the above mentioned further data in order to evaluate the efficacy of a drug (for example: does the drug reach its target?), to evaluate whether it is necessary to re-administer the drug (for example: is the drug still present at the target or not?) etc.

“Organ” includes in this regard one or more organs selected from the digestive system (including salivary glands, esophagus, stomach, liver, gallbladder, pancreas, intestines, rectum and anus); endocrine system (including endocrine glands such as the hypothalamus, pituitary or pituitary gland, pineal body or pineal gland, thyroid, parathyroids and adrenals, i.e., adrenal glands); integumentary system (including skin, hair and nails); lymphatic system (including lymphatic system, lymph nodes, tonsils, adenoids, thymus and spleen); muscular system; nervous system (including brain, spinal cord, peripheral nerves and nerves); reproductive system (including ovaries, fallopian tubes, uterus, vagina, mammary glands, testes, vas deferens, seminal vesicles, prostate and penis); respiratory system (including the pharynx, larynx, trachea, bronchi, lungs and diaphragm); skeletal system (including bones, cartilage, ligaments and tendons); and the urinary system (including kidneys, ureters, bladder and urethra involved in fluid balance, electrolyte balance and excretion of urine).

The heart, liver, kidneys, spleen, bladder, lungs and brain are preferred; bladder and/or liver being even more preferred.

The present invention also relates to the method of the present invention, wherein in (b) said light with a wavelength distinguishable from the predetermined wavelength of (a) is received with an optical detector.

The term “optical detector” includes any suitable light detection or image recording system which is able to convert light energy or other electromagnetic energy into a measurable electrical signal. Optical detectors are sometimes also termed photodetector or photosensor. An optical detector can be exemplified as a charge coupled device (CCD), a photodiode, a photoconductive cell, a complementary metal oxide semiconductor (CMOS), photomultiplier tube, a photoresistor, a phototransistor, a reverse-biased LED, or as a cryogenic detector. CCD and CMOS are preferred.

It is envisaged that the optical detector is or comprises an imaging device which can optionally include a lense system and/or a camera. Additionally, it is also envisaged that the imaging device includes features to increase sensitivity to detect the emitted/emission light, such as, image intensifiers, large on-chip microlenses, that reduce the inefficient area of the chip, and improve overall quantum-efficiency. Alternatively, thinned back illuminated and cooled CCDs or CCDs with image intensifiers can be used. The concentration and quantum efficiency of the fluorophores in the target region of the biological tissue is an additional factor that affects sensitivity. A way to improve sensitivity is by developing fluorophores with improved quantum efficiency, as well as with the use of less-quenching fluorophores. Moreover, as fluorophores are used with excitation and emission spectra spaced further apart, the pass bands of an optional bandpass filters can be broadened to collect a greater percentage of the respective fluorescent photons without increasing crosstalk. All these measure are well-known to the skilled person and exemplified for example in WO 2005/062987.

The imaging device may further comprise an image capture processor adapted to capture a plurality of images from the optical detector. Each image within the plurality of images has a respective exposure time. The image capture processor can include an exposure processor adapted to dynamically adjust the respective exposure times associated with each image within the plurality of images. The imaging system may further include display and storage means, for example a memory coupled to the image capture processor and adapted to receive and store the plurality of images and adapted to receive and store the respective exposure times.

The imaging device may further comprise image-processing software which enables generation of calculated images. For example, real-time or near real-time image streams are displayed as overlay, false-colored images, subtraction images, or division images. Other mathematical functions can be used to process the images, including noise-filtering techniques, ratio imaging, threshold detection and/or prior probability analysis to facilitate the detection of biological information.

The optical detector which may be used in the context of the present invention may optionally (i) comprise a pre-determined or tunable filter (hardware filter) which is preferably upstream of said detector, and/or (ii) may separate (software filter) the predetermined wavelength of the excitation light, used in step (a) of the methods of the invention (i.e. in the step of directing the excitation light to the delineated region as described herein).

Optical filters selectively transmits light having certain properties (often, a particular range of wavelengths), while blocking the remainder. Said properties are fixed in a pre-determined filter and alterable in a tunable filter. The wavelength bands that are transmitted and/or reflected in the optical imaging system can be tuned, for example, by a change in the angle of incidence of the incoming beam. Selection of this incidence angle enables fine-tuning of the spectral band that is transmitted and the spectral band that is reflected.

Said pre-determined or tunable filter is preferably “upstream of said detector”, i.e. it is provided somewhere between the optical detector and the subject.

It is also envisaged to use a “software filter” in combination with a hardware filter or alternatively to a hardware filter. A software filter allows for the separation of the predetermined wavelength of the excitation light, i.e. it is thereby possible to deduct the wavelength of the excitation light.

It is preferred that said filter (hardware) specifically rejects the predetermined wavelength of the excitation light, used in step (a) of the methods of the present invention.

The present invention furthermore relates to methods, wherein step (b) is characterized by the step of receiving light emitted from said fluorescent analyte with a wavelength distinguishable from the predetermined wavelength of (a), exclusively determining the amount of light emitted through the eye of said subject, thereby:
(i) determining the presence of said fluorescent analyte;
(ii) quantifying the blood level of said fluorescent analyte; or
(iii) monitoring or determining the blood clearance of said fluorescent.

In a further embodiment, the present invention relates to the methods as disclosed herein, wherein the optical axis of the/an excitation means which is used to direct the excitation light of at least one predetermined wavelength onto a delineated region comprising at least a portion of the pupil of the subject to excite the fluorescent analyte, and the optical axis of the optical detector which is used to receive the light emitted from said fluorescent analyte with a wavelength distinguishable from the predetermined wavelength, are arranged parallel to one another and/or at an angle to one another. Said arrangement may be fixed or adjustable. It is also envisaged that the excitation means and/or the optical detector are movable. “Excitation means” thereby includes the light source which provides the excitation light (and optionally a filter).

The methods of the present invention further encompass embodiments wherein the optical axis of the excitation light(s) is fully identical with, is not identical with, or only at part identical with the optical axis of the emission light. It is thus also envisaged that the optical axis of the excitation light(s) is at an angle to the optical axis of the emission light.

The present invention also envisages methods, wherein the excitation means which is used to direct the excitation light of at least one predetermined wavelength onto a delineated region comprising at least a portion of the pupil of the subject to excite the fluorescent analyte, and the optical detector which is used to receive the light emitted from said fluorescent analyte with a wavelength distinguishable from the predetermined wavelength, are spatially separated or unified (a unit).

The following, non-limiting, list of technical fields illustrates the broad applicability of the present invention. The methods and/or devices of the present invention may be used for:
(a) determining the presence of an analyte in the blood of a subject;
(b) determining the biological half-life (t1/2) of analyte in the blood of a subject;
(c) quantifying the blood level of an analyte in the blood of a subject,
(d) monitoring or determining the blood clearance of an analyte in the blood of a subject;
(e) determining the serum peak level (tmax) of an analyte in the blood of a subject;
(f) determining the theoretical concentration of an analyte in the blood of a subject;
(g) evaluating saturation kinetics in the blood of a subject, thereby, for example, determining the amount of an antibody bound to, for example, a tumor;
(h) determining the dissolution kinetic of a pharmaceutical or diagnostic composition in the blood of a subject;
(i) evaluating the elimination kinetic and/or pathway of an analyte in the blood of a subject;
(j) evaluating the pharmacokinetics of an analyte in the blood of a subject;
(k) determining bioavailability of an analyte in the blood of a subject. Bioavailability is the percentage or fraction of the administered dose of an analyte that reaches the systemic circulation of a subject. Examples of factors that can alter bioavailability include inherent dissolution and absorption characteristics of the administered drug (e.g., salt, ester), the dosage form (e.g., tablet, capsule), the route of administration, the stability of the active ingredient in the gastrointestinal tract, and the extent of drug metabolism before reaching the systemic circulation;
   and so forth.

Further applications of the methods/devices of the present invention are exemplified herein elsewhere. The term “analyte” equates in this context with fluorescent analyte, second entity, target etc. which are described herein.

The present invention also envisages a fluorescent analyte or fluorescent label as defined herein for use in the methods of the present invention.

The present invention furthermore relates to the use of a fluorescent analyte or fluorescent label as defined herein for the preparation of a pharmaceutical and/or diagnostic composition which is, preferably, to be employed in the methods of the invention.

The “pharmaceutical or diagnostic composition” may comprise the fluorescent analyte, second entity, fluorescent label, target etc. of the invention and, optionally a pharmaceutically or diagnostically acceptable carrier and/or diluent.

Examples of suitable carriers and/or diluents are well known in the art and include phosphate buffered saline solutions, water, emulsions, such as oil/water emulsions, various types of wetting agents, sterile solutions etc. Compositions comprising such carriers can be formulated by well known conventional methods.

In a preferred embodiment, said device comprises:
(a) excitation means to direct excitation light of at least one predetermined wavelength onto a delineated region comprising at least a portion of the pupil of the subject to excite the fluorescent analyte; and
(b) an optical detector to receive exclusively the light emitted from said fluorescent analyte with a wavelength distinguishable from the predetermined wavelength of (a).

In an alternative embodiment, said device comprises:
(a) excitation means to direct excitation light of at least one predetermined wavelength exclusively onto a delineated region comprising at least a portion of the pupil of the subject to excite the fluorescent analyte; and
(b) an optical detector to receive the light emitted from said fluorescent analyte with a wavelength distinguishable from the predetermined wavelength of (a).

The present invention also relates to a device comprising:
(a) excitation means to direct excitation light of at least one predetermined wavelength exclusively onto a delineated region comprising at least a portion of the pupil of the subject to excite the fluorescent analyte; and
(b) an optical detector to receive exclusively the light emitted from said fluorescent analyte with a wavelength distinguishable from the predetermined wavelength of (a).

The present invention further relates to a device comprising:
(a) excitation means to direct excitation light of at least one predetermined wavelength onto a delineated region comprising at least a portion of the pupil of the subject to excite the fluorescent analyte; and
(b) an optical detector to receive the light emitted from said fluorescent analyte with a wavelength distinguishable from the predetermined wavelength of (a); and
(c) means to determine exclusively the amount of light emitted through the eye of said subject.

In a preferred embodiment of the devices of the present invention, said optical detector comprises a pre-determined or tunable filter which is connected upstream of said detector. Preferably, said filter rejects the at least one predetermined wavelength of the excitation light.

In a further preferred embodiment of the devices of the present invention, said optical detector separates the predetermined wavelength of the excitation light, used in (a).

It is also envisaged that the optical axis of said excitation means, and the optical axis of the optical detector which is used to receive the light emitted from said fluorescent analyte with a wavelength distinguishable from the predetermined wavelength of (a), are arranged parallel to one another and/or at an angle to one another.

In a further embodiment of the devices of the invention said excitation means and said optical detector are spatially separated or unified (i.e. they form a unit). It is also envisaged that said excitation means and/or said optical detector is/are movable.

The devices of the present invention may optionally comprise means to determine:
(i) the location of the pupil of the eye; and/or
(ii) the area of said portion of the pupil in step (a), and/or the area of the pupil of said eye in step (b).

It is envisaged that the device of the present invention is or comprises an eyeglass.

It is preferred that the devices of the present invention have, as such, no direct contact with the cornea of the eye of a subject. It is particularly preferred that the devices of the present invention are not formed as contact lenses.

This disclosure may best be understood in conjunction with the accompanying drawings, incorporated herein by references. Furthermore, a better understanding of the present invention and of its many advantages will be had from the following examples, given by way of illustration and are not intended as limiting.

### FIGURES

The figures show:

FIG. 1 Theoretical example to illustrate that the determination of tmax and t1/2 by interpolation, may influence the accuracy of these values.

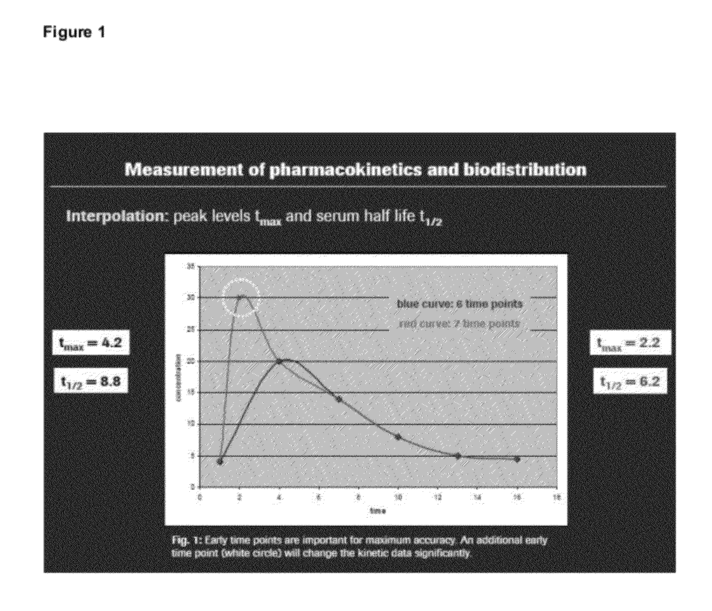

FIG. 2 Theoretical example to illustrate that the determination of tmax and t1/2 by interpolation, may influence the accuracy of these values.

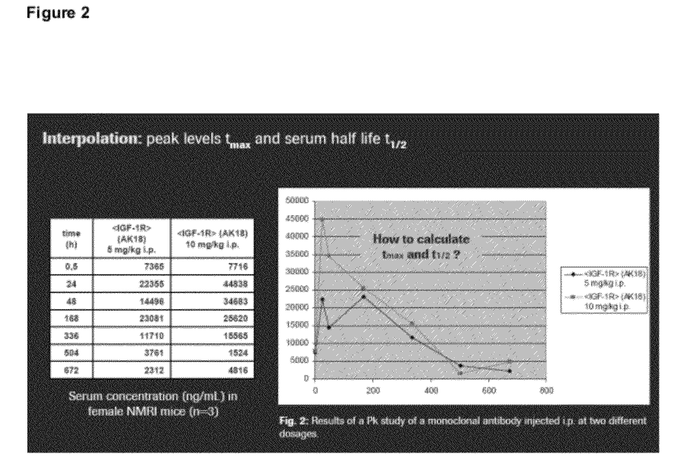

FIG. 3 Optical imaging equipment

Representative example of the optical imaging equipment of the present invention. Anesthetized mice are placed in the imaging chamber (1), injected with the labeled drug and illuminated with light of a certain wavelength (2). The light radiated back from the fluorophores in the object under examination (3), passes through an emission filter (4) before being detected by the CCD camera (5). The resulting image is displayed on the PC as a grayscale (6) or pseudo color image, depending on the selected wavelength, and can be further processed (7).

FIG. 4 Monitoring the fluorescence intensity of ICG in the eye of a mouse

Female BALB/c nude received inhalation anesthesia, were placed into the imaging chamber and injected i.v. with ICG (20 μg/200 μl). The fluorescence signal intensity was measured starting 10 sec before injection of ICG. Images in the region of interest were recorded every second with an acquisition time of 500 ms over a period of 8 min. ICG was excited with light at a wavelength range from 671 to 705 nm and the emission was detected at 820 nm. The fluorescence intensity from these regions were then calculated and plotted as a function of time.

FIG. 5 Facilitated calculation of tmax and t1/2 without interpolation of data

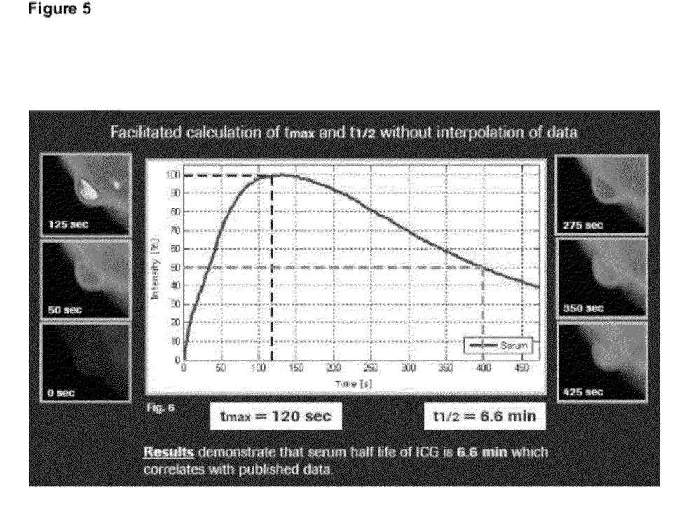

FIG. 6 Monitoring the fluorescence intensity of ICG in different mouse organs

The fluorescence signal intensity in Calu3 xenograft was measured as described before. ROI were identified for the eye, liver, kidney, brain and s.c. growing tumor using the anatomical pictures of the subjects. The fluorescence intensity from these regions were then calculated and plotted as a function of time.

FIG. 7 Monitoring fluorescence intensity of Pamidronate in the whole mouse

A fluorescence labeled bisphosphonate (OsteoSense; VisenMedical, Woburn, USA) was injected i.v. (2 nMol in 200 μl PBS) and fluorescence signal intensity was recorded over a time period of 4.4 hours (acquisition time: 3000 ms; excitation wavelength: 615 to 665 nm; emission wavelength: 780 nm). The fluorescence intensity in the eye was calculated and plotted as a function of time.

FIG. 8 Facilitated calculation of tmax and t1/2 without interpolation of data

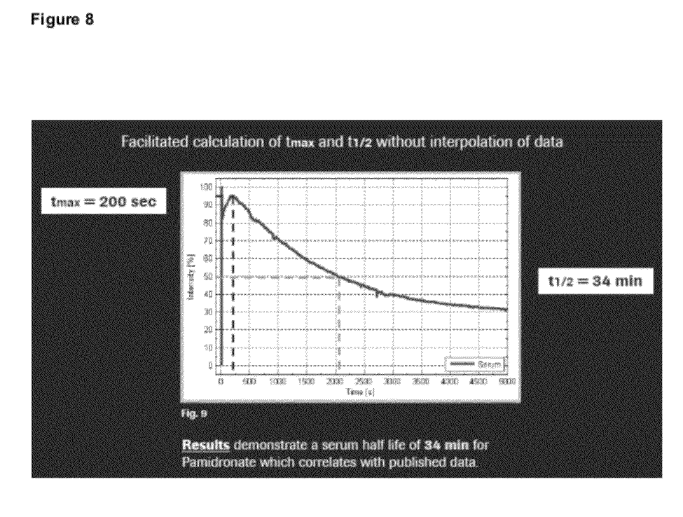

FIG. 9 Confirmation of accumulation of Pamidronate in target organs

Mouse was injected i.v. with 2 mmol OsteoSense750 and NIRF was measured 48 h thereafter.

FIG. 10 Conventional PK measurement of anti-receptor tyrosine kinase Ab

FIG. 11 Monitoring the fluorescence intensity of labeled anti-RTK Ab in the eye of a mouse

A Cy5 labeled mAb against receptor tyrosine kinase was injected i.v. (2.5 mg/kg) and fluorescence signal intensity was recorded over a time period of 3 hours (acquisition time: 500 ms; excitation wavelength: 615 to 665 nm; emission wavelength: 720 nm). The fluorescence intensity in the eye was calculated and plotted as a function of time.

### EXAMPLES

The following examples illustrate the invention. These examples should not be construed as to limit the scope of this invention. The examples are included for purposes of illustration and the present invention is limited only by the claims.

We propose a method which allows continuous monitoring of drug levels in plasma/serum and measurements of organ distribution simultaneously in one mouse over a time period of at least 4 hours. We demonstrate the utility of this approach by evaluating 3 different compounds:

1. Indocyanine green: a fluorescent dye
2. Pamidronate: a fluorescence labeled bisphosphonate
3. Monoclonal antibody against receptor tyrosine kinase labeled with Cy5

### Example 1

### Feasibility Study with Indocyanine Green (ICG)

We first evaluated the technical feasibility of this new approach by using indocyanine green (ICG) a fluorescent dye. When injected i.v. into mice, ICG is cleared from the circulation in approximately 2 to 4 min (1, 2) and accumulates in the liver (3).

### Material and Methods

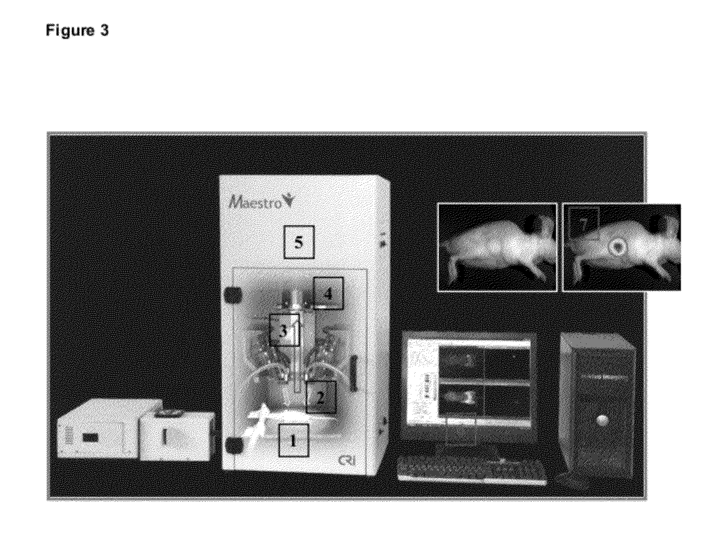

Female BALB/c nude mice received inhalation anesthesia, were placed in the imaging chamber (FIG. 3) and injected i.v. with a dose of 20 μg/200 μl. The fluorescence signal intensity measurements in the eye was started 10 sec before i.v. injection of ICG and images were recorded every second with an acquisition time of 500 ms over a period of 8 minutes. ICG was excited with light at a wavelength range from 671 to 705 nm and the emission was detected at 820 nm.

### Results

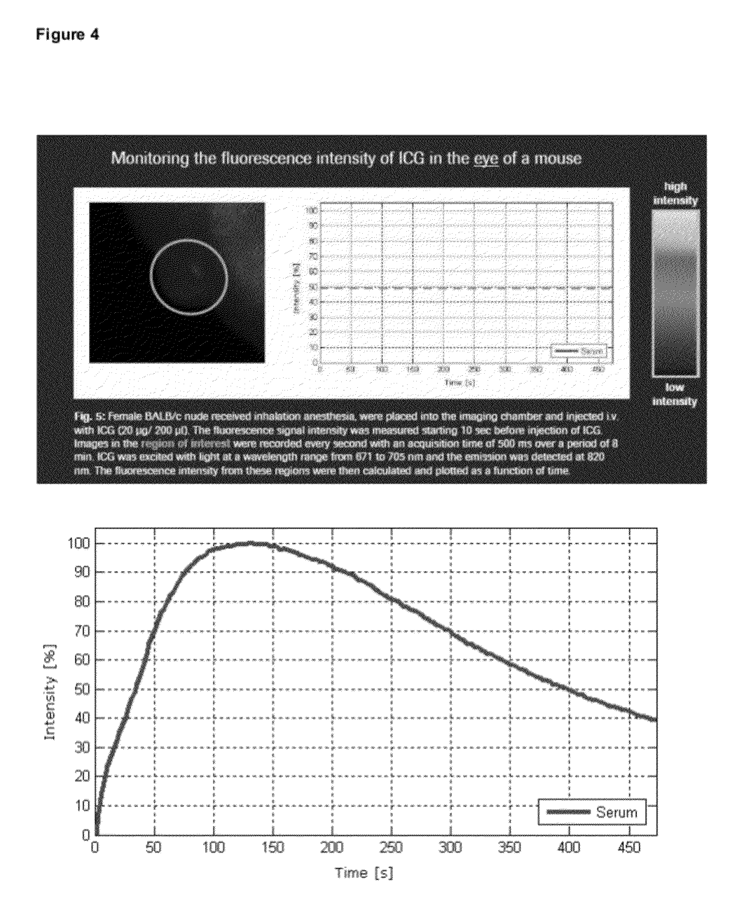

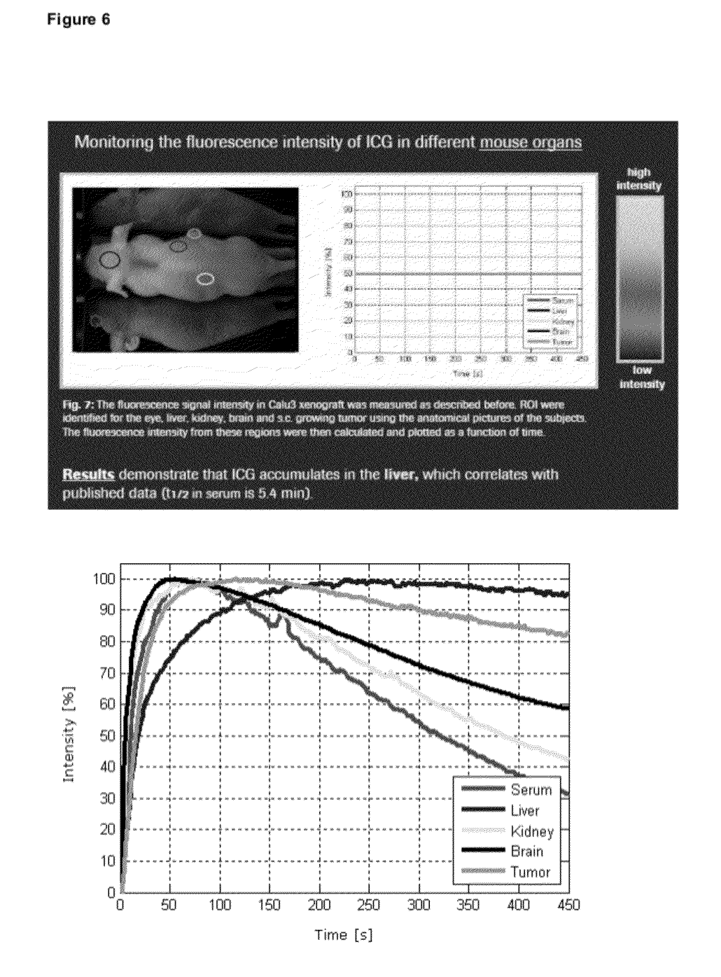

The highest value of the fluorescence signal intensity in the eye region was normalized to 100 and data depicted in FIG. 4, 5 demonstrate that tmax was reached at 2 min and the half life of the fluorescence intensity was at 6.6 min. In a second experiment, a BALB/c nude mouse with a s.c. growing tumor (Calu3) was injected with ICG i.v. and signal intensity in eye, liver, kidney, brain and the tumor region was monitored. In this experiment tmax was 1.2 min. The signal intensity declined thereafter (t1/2=5.4 min) and accumulation in the liver was observed reaching a plateau at 3.8 min (FIG. 6). These results are in accordance with published data.

### Example 2

### Feasibility Study with Pamidronate, a Fluorescence Labeled Bisphosphonate

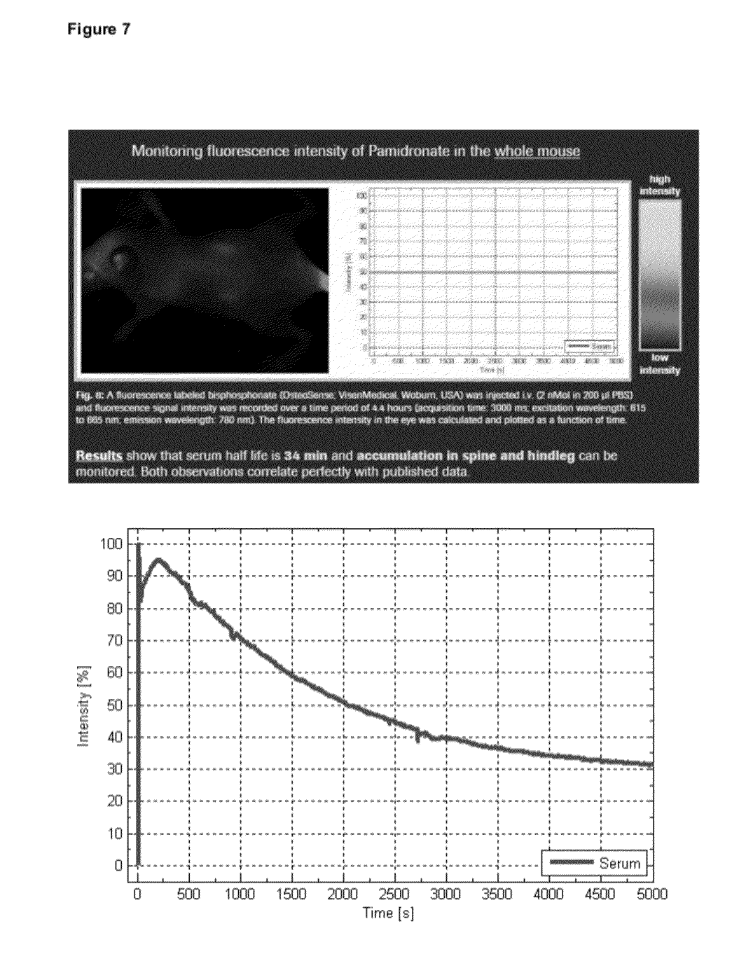

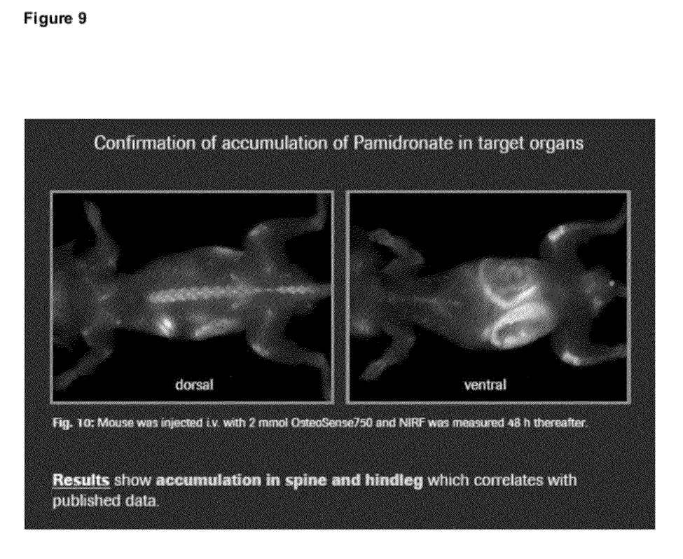

After successful completion of the feasibility study shown in Example 1, we evaluated a fluorescence labeled bisphosphonate (Pamidronate). Bisphosphonates (e.g. Pamidronate; MW 279) are clinically useful for the treatment of bone disorders. Pamidronate (after i.v. injection) has a serum half life in the range of 20 to 30 min and the bone (tibia) contained the highest concentration of all the tissues examined. Pongchaidecha M et al. Clearance and tissue uptake following 4-hour and 24-hour infusions of pamidronate in rats. Drug Metab Dispos 1993; 21(1):100-104 Daley-Yates et al. A comparison of the pharmacokinetics of 14C-labelled ADP and 99 mTc-labelled ADP in the mouse. Calcif Tissue Int 1988; 43:125-127. We used a fluorescence labeled Pamidronate to calculate tmax and t1/2 plasma levels by measuring the fluorescence intensity in the eye of mice and whole body imaging to monitor the described kinetics. After i.v. injection, Pamidronate has a serum half life in the range of 20 to 30 min and the bone (tibia) contained the highest concentration of all the tissues examined (4, 5). OsteoSense (2 nMol in 200 μl PBS) was injected i.v. and fluorescence signal intensity was recorded (acquisition time: 3000 ms; excitation wavelength: 615 to 665 nm; emission wavelength: 780 nm). Serum t1/2 was 34 min (FIG. 7, 8) and accumulation in spine and hind leg is clearly demonstrated at 4.4 hrs and 48 hrs thereafter (FIG. 9). Both observations correlate with published data.

### Example 3

### Feasibility Study with a Non-Labeled and Cy5 Labeled Monoclonal Antibody Targeting Receptor Tyrosine Kinase

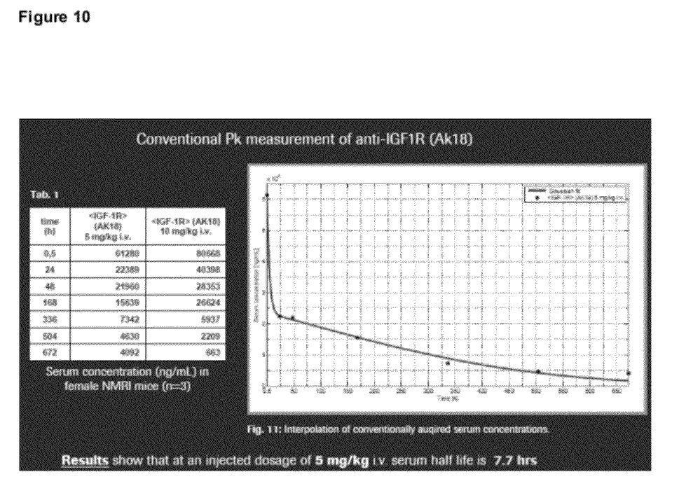

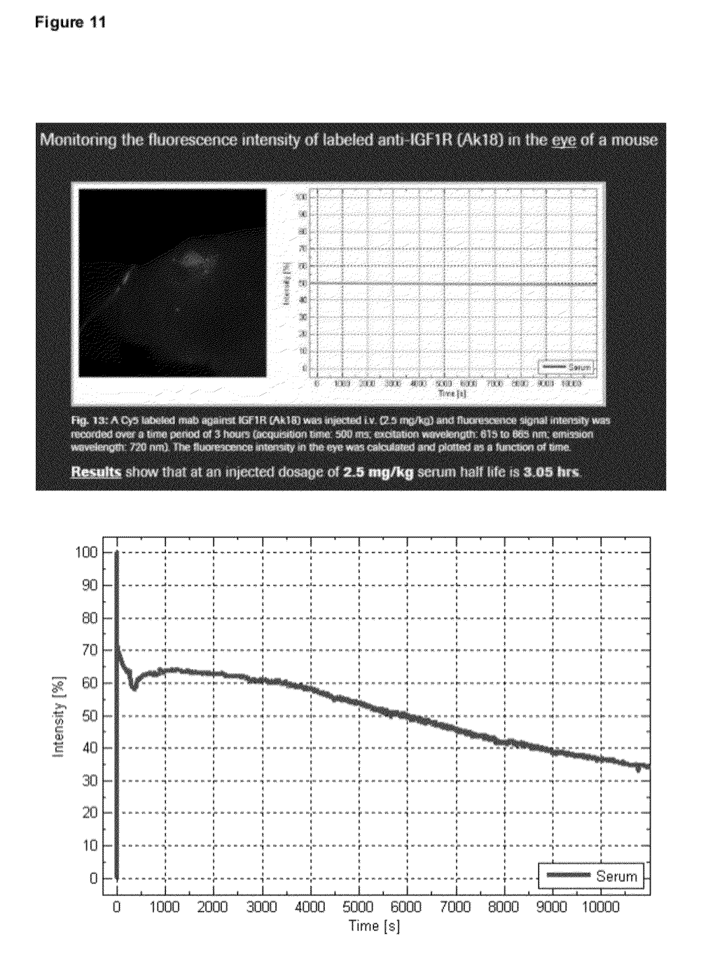

Finally, t1/2 of a non-labeled and Cy5 labeled monoclonal antibody targeting receptor tyrosine kinase was compared. Conventional measurements revealed a t1/2 of 7.7 hrs at a dosage of 5 mg/k i.v. (FIG. 10). Using optical imaging t1/2 was 3.05 hrs at a dosage of 2.5 mg/kg i.v. (FIG. 11).

### Discussion

These results demonstrate that tmax and t1/2 can be easily performed by simply measuring the fluorescence signal intensities in the eye of anesthesized animals. In contrast to the conventional technique this new approach improves the performance of PK studies since quantification of the drug and data interpolation is not necessary. Furthermore, the number of mice is significantly reduced and mice need not to be sacrificed. Information regarding the accumulation of the drug and t1/2 values from different organs can be obtained time-resolved and on-line.

Taking together, this procedure allows multiple measurements in one animal (improving the accuracy of the tmax and t1/2). Compared to conventional methods, work time is significantly reduced, mixing up of blood samples is prevented and the use of non-radioactive materials permits further analysis by routine laboratory methods without the precautions needed with radiochemicals. In addition to tmax and t1/2, organ distribution can be followed up. Such simultaneous measurements facilitates information regarding accumulation in the organ under question compared with t1/2 in serum (e.g. indication of blood brain barrier penetration). Drugs (low molecular weight substances, peptides, proteins, antibodies and siRNA) can be labeled easily with different organic fluorescence dyes. However, before performing such in vivo studies with labeled drugs, functional assays must demonstrate that there is no difference compared to the non-labeled drug.

Regarding Hemojuvelin, in vitro studies confirmed that non-labeled and Cy5-labeled Hemojuvelin did not differ in their ability to block BMP-2 induced upregulation of Hepcidin mRNA in HepG2 cells. Also, Biacore data reveal that Cy5-labeled Herceptin has the same binding characteristics compared to non-labeled Herceptin and binds to Her2 expressing tumor cells. The labeled antibody targeting receptor tyrosine kinase still leads to internalization of the receptor.

Since animals are not sacrificed, multiple applications of the same and/or another drug (labeled with a fluorochrome with a emission spectra different from the first one) can be applied to get information on drug-drug interactions. Furthermore, new designed drug formulations and optimization of drug dosage after i.v., i.p., oral, inhalation, nasal and dermal applications can be evaluated in normal and in genetically engineered mice (e.g. FcRn knock-outs or hu FcRn transgenics).

The extraordinary progress of imaging methods allows the visualization of the performance of drugs and drug delivery systems under in vivo conditions. Detailed and quantitative information about the location and concentration of the drug can be obtained as a function of time, thereby enabling a more profound understanding of biological effects. This information is crucial to the design of optimized drugs.

### REF

1. Desmettre T et al. Fluorescence properties and metabolic features of indocyanine green (10G) as related to angiography. Sury Ophthalmol 2000; 45:15-27.
2. Saxena V et al. Polymeric nanoparticulate delivery system for Indocyanine green: Biodistribution in healthy mice. International Journal of Pharmaceutics 2006; 308: 200-204.
3. Paumgartner G. The handling of indocyanine green by the liver. Schweiz Med. Wochenschr. 1975; 105(17 Suppl):1-30.
4. Pongchaidecha M et al. Clearance and tissue uptake following 4-hour and 24-hour infusions of pamidronate in rats. Drug Metab Dispos 1993; 21(1):100-104
5. Daley-Yates et al. A comparison of the pharmacokinetics of 140-labelled ADP and 99 mTc-labelled ADP in the mouse. Calcif Tissue Int 1988; 43:125-127.

### Further Items of the Invention are:

1. A non-invasive method of monitoring or determining the blood clearance of a fluorescent analyte which comprises a fluorescent entity and a second entity, in a subject, comprising the steps of:
   (a) directing excitation light of at least one predetermined wavelength onto a delineated region comprising at least a portion of the pupil of said subject, to excite the fluorescent entity,
   (b) receiving light emitted from said fluorescent analyte with a wavelength distinguishable from the predetermined wavelength of (a), through the eye of said subject, thereby monitoring or determining the blood clearance of said fluorescent analyte.
2. A non-invasive method of quantifying the blood level of a fluorescent analyte which comprises a fluorescent entity and a second entity, in a subject, comprising the steps of:
   (a) directing excitation light of at least one predetermined wavelength onto a delineated region comprising at least a portion of the pupil of said subject, to excite the fluorescent entity,
   (b) receiving light emitted from said fluorescent analyte with a wavelength distinguishable from the predetermined wavelength of (a), through the eye of said subject, thereby quantifying the blood level of a fluorescent analyte.
3. A non-invasive method of determining the presence of a fluorescent analyte which comprises a fluorescent entity and a second entity, in the blood of a subject, comprising or consisting of the steps:
   (a) directing excitation light of at least one predetermined wavelength onto a delineated region comprising at least a portion of the pupil of said subject, to excite the fluorescent entity,
   (b) receiving light emitted from said fluorescent analyte with a wavelength distinguishable from the predetermined wavelength of (a), through the eye of said subject, thereby determining the presence of said fluorescent analyte in the blood of said subject.
4. The method of any one of items 1 or 2, wherein said light received in step (b) is compared with a reference value, thereby:
   (i) quantifying the blood level of said fluorescent analyte; or
   (ii) determining the blood clearance of said fluorescent analyte.
5. The method of any one of items 1 to 4, wherein said second entity comprises a diagnostic and/or therapeutic agent.
6. The method of any one of the preceding items, wherein said second entity comprises at least one epitope binding domain which is specific for a target.
7. The method of item 6, wherein a fluorescent label comprised in said fluorescent entity is activated once the epitope binding domain of said second entity has bound to its target.
8. The method of item 6, wherein said target is or comprises an antibody or a functional fragment thereof, a protein, a peptide, an enzyme, a nucleic acid for example siRNA, a polysaccharide, a lipid, a receptor, a receptor ligand, a pathogen, a virus, a bacterium, a cell, a cellular target, a tumor antigen and/or a drug.
9. The method of any one of the preceding items, wherein said second entity is directly labeled with said fluorescent entity.
10. The method of any one of the preceding items wherein said second entity is indirectly labeled with said fluorescent entity.
11. The method of item 10, wherein said fluorescent entity comprises a epitope binding domain which is specific for said second entity.
12. The method of any one of the preceding items, wherein the fluorescent entity is activatable.
13. The method of any one of items 10 to 12, wherein said fluorescent entity is activated once it has bound to the second entity.
14. The method of item 13, wherein said fluorescent entity is to be activated in said subject.
15. The method of item 14, wherein said activation precedes step (b).
16. The method of any one of items 1 to 4, wherein the fluorescent analyte is the fluorescent entity.
17. The method of any one of the preceding items, wherein said fluorescent analyte is to be administered to said subject.
18. The method of item 11, wherein said fluorescent entity is to be administered to said subject.
19. The method of item 8, wherein said target is to be administered to said subject.
20. The method of any one of items 17 to 19, wherein said administration of said fluorescent analyte, said fluorescent entity and/or said target precedes step (b).
21. The method of any one of the preceding items, wherein step (b) is characterized by the step of receiving light emitted from said fluorescent analyte with a wavelength distinguishable from the predetermined wavelength of (a), exclusively determining the amount of light emitted through the eye of said subject, thereby:
   (i) determining the presence of said fluorescent analyte;
   (ii) quantifying the blood level of said fluorescent analyte; or
   (iii) monitoring or determining the blood clearance of said fluorescent
22. The method of any one of the preceding items, wherein said light which is emitted from said fluorescent analyte with a wavelength distinguishable from the predetermined wavelength of (a), is exclusively received through the eye of said subject.
23. The method of any one of the preceding items, wherein in (a) said excitation light of at least one predetermined wavelength is exclusively directed onto a delineated region comprising at least a portion of the pupil of said subject.
24. The method of any one of the preceding items, further comprising step (c) determining light emitted from said fluorescent analyte with a wavelength distinguishable from the predetermined wavelength of (a), from further regions of the subject.
25. The method of item 24, wherein the further regions comprise at least a part of an organ, lymph nodes, blood vessel networks or nervous cell system.
26. The method of item 25, wherein said organ is selected from the group consisting of the heart, liver, kidneys, spleen, bladder, lungs and brain, bladder and/or liver being preferred.
27. The method of any one of the preceding items, wherein the subject is an animal, preferably a human.
28. The method of any one of the preceding items, wherein in (b) said light with a wavelength distinguishable from the predetermined wavelength of (a) is received with an optical detector.
29. The method of item 28, wherein said optical detector:
   (i) comprises a pre-determined or tunable filter upstream of said detector, and/or
   (ii) separates the predetermined wavelength of the excitation light, used in (a).
30. The method of item 29, wherein said filter rejects the predetermined wavelength of the excitation light, used in (a).
31. The method of any one of the preceding items, wherein the optical axis of an excitation means which is used to direct the excitation light of at least one predetermined wavelength onto a delineated region comprising at least a portion of the pupil of the subject to excite the fluorescent analyte, and the optical axis of the optical detector which is used to receive the light emitted from said fluorescent analyte with a wavelength distinguishable from the predetermined wavelength, are arranged at an angle to each other.
32. The method of any one of the preceding items, wherein the excitation means which is used to direct the excitation light of at least one predetermined wavelength onto a delineated region comprising at least a portion of the pupil of the subject to excite the fluorescent analyte, and the optical detector which is used to receive the light emitted from said fluorescent analyte with a wavelength distinguishable from the predetermined wavelength, are spatially separated.
33. The method of any one of the preceding items, wherein said optical detector is or comprises a photodiode, a photoconductive cell, a charge coupled device (CCD), or a complementary metal oxide semiconductor (CMOS).
34. The method of any one of the preceding items which is for determining the presence, quantifying the blood level, monitoring or determining the blood clearance of a drug, an antibody or a functional fragment thereof, a protein, a peptide, an enzyme, a nucleic acid for example siRNA, a polysaccharide, a lipid, a receptor, a receptor ligand, a pathogen, a virus, a bacterium, a cell, a cellular target, and/or a tumor antigen in the blood of a subject, for determining the dissolution kinetic of a pharmaceutical or diagnostic composition, and/or for evaluating the elimination pathway of a substance.
35. The method of any one of the preceding items, wherein steps (a) and/or (b) further include determining the location of the pupil of the eye.
36. The method of any one of the preceding items, further comprising:
   (i) determining the area of said portion of the pupil in step (a), and/or
   (ii) determining the area of the pupil of said eye in step (b).
37. The fluorescent label as defined in any one of the preceding items, which is selected from the group consisting of quantum dot agents, fluorescent dyes, pH-sensitive fluorescent dyes, voltage sensitive fluorescent dyes, and fluorescent labeled microspheres.
38. A fluorescent analyte or fluorescent label as defined in any one of the preceding items for use in any one of the preceding methods.
39. Use of a fluorescent analyte or fluorescent label as defined in any one of the preceding items for the preparation of a diagnostic composition which is to be employed in any one of the preceding methods.
40. A device for use in any of the methods defined in the preceding items.
41. A device for use in any of the above defined methods, which comprises:
   (a) excitation means to direct excitation light of at least one predetermined wavelength onto a delineated region comprising at least a portion of the pupil of the subject to excite the fluorescent analyte; and
   (b) an optical detector to receive exclusively the light emitted from said fluorescent analyte with a wavelength distinguishable from the predetermined wavelength of (a).
42. A device for use in any of the above defined methods, which comprises:
   (a) excitation means to direct excitation light of at least one predetermined wavelength exclusively onto a delineated region comprising at least a portion of the pupil of the subject to excite the fluorescent analyte; and
   (b) an optical detector to receive the light emitted from said fluorescent analyte with a wavelength distinguishable from the predetermined wavelength of (a).
43. A device for use in any of the above defined methods, which comprises:
   (a) excitation means to direct excitation light of at least one predetermined wavelength exclusively onto a delineated region comprising at least a portion of the pupil of the subject to excite the fluorescent analyte; and
   (b) an optical detector to receive exclusively the light emitted from said fluorescent analyte with a wavelength distinguishable from the predetermined wavelength of (a).
44. A device for use in any of the above defined methods, which comprises:
   (a) excitation means to direct excitation light of at least one predetermined wavelength onto a delineated region comprising at least a portion of the pupil of the subject to excite the fluorescent analyte; and
   (b) an optical detector to receive the light emitted from said fluorescent analyte with a wavelength distinguishable from the predetermined wavelength of (a); and
   (c) means to determine exclusively the amount of light emitted through the eye of said subject.
45. The device of any one of items 41 to 44, wherein said optical detector comprises a pre-determined or tunable filter which is connected upstream of said detector.
46. The device of any one of items 41 to 45, wherein said optical detector separates the predetermined wavelength of the excitation light, used in (a).
47. The device of item 45, wherein said filter rejects the at least one predetermined wavelength of the excitation light, used in (a).
48. The device of any one of items 41 to 47, wherein the optical axis of said excitation means, and the optical axis of the optical detector which is used to receive the light emitted from said fluorescent analyte with a wavelength distinguishable from the predetermined wavelength of (a), are arranged at an angle to each other.
49. The device of any one of the preceding items, wherein said excitation means and said optical detector are spatially separated.
50. The device of item 49, wherein said excitation means and/or said optical detector is/are movable.
51. The device of any one of the preceding items, further comprising means to determine:
   (i) the location of the pupil of the eye; and/or
   (ii) the area of said portion of the pupil in step (a), and/or the area of the pupil of said eye in step (b).
52. The device of any one of the preceding items, wherein said device is or comprises an eyeglass.

1. A non-invasive method of monitoring or determining the blood clearance of a fluorescent analyte which comprises a fluorescent entity and a second entity, in a subject, comprising the steps of:
(a) directing excitation light of at least one predetermined wavelength onto a delineated region comprising at least a portion of the pupil of said subject, to excite the fluorescent entity,
(b) receiving light emitted from said fluorescent analyte with a wavelength distinguishable from the predetermined wavelength of (a), through the eye of said subject, thereby monitoring or determining the blood clearance of said fluorescent analyte.

2. A non-invasive method of quantifying the blood level of a fluorescent analyte which comprises a fluorescent entity and a second entity, in a subject, comprising the steps of:
(a) directing excitation light of at least one predetermined wavelength onto a delineated region comprising at least a portion of the pupil of said subject, to excite the fluorescent entity,
(b) receiving light emitted from said fluorescent analyte with a wavelength distinguishable from the predetermined wavelength of (a), through the eye of said subject, thereby quantifying the blood level of a fluorescent analyte.

3. A non-invasive method of determining the presence of a fluorescent analyte which comprises a fluorescent entity and a second entity, in the blood of a subject, comprising or consisting of the steps:
(a) directing excitation light of at least one predetermined wavelength onto a delineated region comprising at least a portion of the pupil of said subject, to excite the fluorescent entity,
(b) receiving light emitted from said fluorescent analyte with a wavelength distinguishable from the predetermined wavelength of (a), through the eye of said subject, thereby determining the presence of said fluorescent analyte in the blood of said subject.

4. The method of any one of claim 1 or 2, wherein said light received in step (b) is compared with a reference value, thereby:
(i) quantifying the blood level of said fluorescent analyte; or
(ii) determining the blood clearance of said fluorescent analyte.

5. The method of any one of claims 1 to 4, wherein said second entity comprises a diagnostic and/or therapeutic agent.

6. The method of any one of the preceding claims, wherein said second entity comprises at least one epitope binding domain which is specific for a target.

7. The method of any one of the preceding claims, wherein step (b) is characterized by the step of receiving light emitted from said fluorescent analyte with a wavelength distinguishable from the predetermined wavelength of (a), exclusively determining the amount of light emitted through the eye of said subject, thereby:
(i) determining the presence of said fluorescent analyte;
(ii) quantifying the blood level of said fluorescent analyte; or
(iii) monitoring or determining the blood clearance of said fluorescent

8. The method of any one of the preceding claims, wherein said light which is emitted from said fluorescent analyte with a wavelength distinguishable from the predetermined wavelength of (a), is exclusively received through the eye of said subject.

9. The method of any one of the preceding claims, wherein in (a) said excitation light of at least one predetermined wavelength is exclusively directed onto a delineated region comprising at least a portion of the pupil of said subject.

10. The method of any one of the preceding claims, further comprising step (c) determining light emitted from said fluorescent analyte with a wavelength distinguishable from the predetermined wavelength of (a), from further regions of the subject.

11. The method of any one of the preceding claims, wherein the optical axis of the excitation light of at least one predetermined wavelength which is directed onto a delineated region comprising at least a portion of the pupil of the subject to excite the fluorescent analyte, and the optical axis of the light emitted from said fluorescent analyte with a wavelength distinguishable from the predetermined wavelength, are arranged at an angle to each other.

12. The method of any one of the preceding claims, wherein the excitation means which is used to direct the excitation light of at least one predetermined wavelength onto a delineated region comprising at least a portion of the pupil of the subject to excite the fluorescent analyte, and the optical detector which is used to receive the light emitted from said fluorescent analyte with a wavelength distinguishable from the predetermined wavelength, are spatially separated.

13. The method of any one of the preceding claims which is for determining the presence, quantifying the blood level, monitoring or determining the blood clearance of a drug, an antibody or a functional fragment thereof, a protein, a peptide, an enzyme, a nucleic acid for example siRNA, a polysaccharide, a lipid, a receptor, a receptor ligand, a pathogen, a virus, a bacterium, a cell, a cellular target, and/or a tumor antigen in the blood of a subject, for determining the dissolution kinetic of a pharmaceutical or diagnostic composition, and/or for evaluating the elimination pathway of a substance.

14. The method of any one of the preceding claims, wherein in (b) said light with a wavelength distinguishable from the predetermined wavelength of (a) is received with an optical detector.

15. The method of claim 14, wherein said optical detector:
(i) comprises a pre-determined or tunable filter upstream of said detector, and/or
(ii) separates the predetermined wavelength of the excitation light, used in (a).

16. The method of claim 15, wherein said filter rejects the predetermined wavelength of the excitation light, used in (a).
